(19) United States
(12) Patent Application Publication
(10) Pub. No.: US 2020/0197494 A1
(43) Pub. Date: Jun. 25, 2020

(54) PEPTIDE COMPOSITIONS AND METHODS OF USE

(71) Applicant: Dauntless Pharmaceuticals, San Diego, CA (US)

(72) Inventors: Olivier Laurent, San Diego, CA (US); Joel F. Martin, San Diego, CA (US); Sveinbjorn Gizurarson, San Diego, CA (US)

(21) Appl. No.: 16/615,362

(22) PCT Filed: May 25, 2018
(86) PCT No.: PCT/US2018/034728

§ 371 Date: Nov. 20, 2019

Related U.S. Application Data
(60) Provisional application No. 62/511,836, filed on May 26, 2017.

(51) Int. Cl. A61K 38/31; A61K 47/38; A61K 47/10; A61K 47/26; A61K 9/00

(52) U.S. Cl. CPC A61K 38/31; A61K 47/38; A61K 9/0043; A61K 47/26; A61K 47/10

(57) ABSTRACT

Provided herein are pharmaceutical compositions containing a therapeutic agent, an alkylglycoside, and pharmaceutically acceptable excipients and use of such compositions in the treatment of various conditions.

### CROSS-REFERENCE TO RELATED APPLICATION

This application claims priority benefit of U.S. Provisional Application No. 62/511,836, filed May 26, 2017, the disclosure of which is herein incorporated by reference in its entirety.

### FIELD

Provided herein are pharmaceutical compositions containing a therapeutic agent, an alkylglycoside, and a pharmaceutically acceptable excipient and use of such compositions in the treatment of various conditions.

### BACKGROUND

Therapeutic peptides have been used to treat a wide variety of diseases. However, for many therapeutic peptides, there are challenges that remain in drug development, and many existing peptide-based drugs have disadvantages associated with their formulation or route of administration.

One of the major challenges is the generally low bioavailability of therapeutic peptides due to their rapid elimination from the circulation through renal filtration, enzymatic degradation and uptake by the reticuloendothelial system.

Shelf-life imposes another major challenge to the development of peptide drugs. Peptides undergo chemical degradation involving covalent modification of the primary structure via bond cleavage or formation. They also undergo physical degradation involving changes in higher-order structure by denaturation and noncovalent aggregation or precipitation. Chemical and physical degradations often result in change of pharmacological effect and potency.

Furthermore, many peptide drugs are formulated for administration by injection using large gauge needles, leading to pain, injection site reactions, and variability of symptoms across dosing intervals.

Thus, there is a need for peptide compositions with improved bioavailability and stability, as well as more favorable administration profile.

### BRIEF SUMMARY

In one aspect, provided is a pharmaceutical composition comprising a therapeutic peptide or a pharmaceutically acceptable salt thereof, an alkylsaccharide, and a pharmaceutically acceptable excipient selected from the group consisting of polyethylene glycol of average molecular weight less than about 360 Dalton, alkylcellulose, hydroxyalkyl cellulose, and hydroxyalkyl alkylcellulose.

In some embodiments, the therapeutic peptide is cyclic. In some embodiments, the therapeutic peptide is a somatostatin analog or a pharmaceutically acceptable salt thereof. In some embodiments, the therapeutic peptide is selected from the group consisting of lanreotide, octreotide, pasireotide, and pharmaceutically acceptable salts of any of the foregoing. In some embodiments, the therapeutic peptide is octreotide or a pharmaceutically acceptable salt thereof.

In some embodiments, the alkylsaccharide is an alkylglycoside. In some embodiments, the alkylsaccharide comprises a C₈-C₁₆ alkyl moiety. In some embodiments, the alkylsaccharide comprises a saccharide selected from the group consisting of maltose, sucrose, and glucose. In some embodiments, the alkylsaccharide consists of a C₈-C₁₆ alkyl (e.g., C₁₂ alkyl) moiety linked by glycosidic linkage to maltose. In some embodiments, the alkylsaccharide is n-Dodecyl-4-O-α-D-glucopyranosyl-β-D-glucopyranoside (DDM).

In some embodiments, the pharmaceutically acceptable excipient is selected from the group consisting of alkylcellulose, hydroxyalkyl cellulose, and hydroxyalkyl alkylcellulose. In some embodiments, the pharmaceutically acceptable excipient is selected from the group consisting of hydroxyethyl cellulose, hydroxylpropyl cellulose, hydroxyethyl methyl cellulose, hydroxypropyl methyl cellulose, ethyl hydroxyethyl cellulose, and methylcellulose. In some embodiments, the pharmaceutically acceptable excipient is Methocel 4000.

In some embodiments, the pharmaceutically acceptable excipient is polyethylene glycol of average molecular weight less than about 360 Dalton. In some embodiments, the average molecular weight of the polyethylene glycol is from 100 to 300 Daltons. In some embodiments, the polyethylene glycol is PEG 200.

In some embodiments, the pharmaceutical composition further comprises a tonicity agent. In some embodiments, the tonicity agent is mannitol.

In some embodiments, the pharmaceutical composition further comprises a buffering agent. In some embodiments, the pH of the composition is from 3 to 8. In some embodiments, the pH is of the composition is from 4.5 to 6. In some embodiments, the buffering agent is a tonicity agent.

In some embodiments, the concentration of the therapeutic peptide (e.g., octreotide, lanreotide, or other somatostatin analog) is from 0.01% (w/w) to 10% (w/w). In some embodiments, the concentration of the therapeutic peptide (e.g., octreotide, lanreotide, or other somatostatin analog) is from 0.1% (w/w) to 3% (w/w). In some embodiments, the concentration of the alkylsaccharide (e.g., DDM) is from 0.01% (w/w) to 5% (w/w). In some embodiments, the concentration of the alkylsaccharide (e.g., DDM) is from 0.1% (w/w) to 1% (w/w). In some embodiments, the concentration of the polyethylene glycol of average molecular weight less than about 360 Dalton, alkylcellulose, hydroxyalkyl cellulose, or hydroxyalkyl alkylcellulose is from 0.01% (w/w) to 5% (w/w). In some embodiments, the concentration of the polyethylene glycol of average molecular weight less than about 360 Dalton, alkylcellulose, hydroxyalkyl cellulose, or hydroxyalkyl alkylcellulose is from 0.01% (w/w) to 1% (w/w). In some embodiments, the concentration of the tonicity agent (e.g., mannitol) is from 0.01% (w/w) to 10% (w/w). In some embodiments, the concentration of the tonicity agent (e.g., mannitol) is from 0.01% (w/w) to 6% (w/w). In any of these embodiments, the pharmaceutical composition may contain a buffering agent.

In another aspect, provided is a method of treating a condition selected from the group consisting of acromegaly, carcinoid tumors, vasoactive intestinal peptide secreting tumors, diarrhea associated with acquired immune deficiency syndrome (AIDS), diarrhea associated with chemotherapy, diarrhea associated with radiation therapy, dumping syndrome, adrenal gland neuroendocrine tumors, bowel obstruction, enterocutaneous fistulae, gastrinoma, acute bleeding of gastroesophageal varices, islet cell tumors, lung neuroendocrine tumors, malignancy, meningiomas, gastrointestinal tract neuroendocrine tumors, thymus neuroendocrine tumors, pancreatic fistulas, pancreas neuroendocrine tumors, pituitary adenomas, short-bowel syndrome, small or large cell neuroendocrine tumors, thymomas and thymic carcinomas, Zollinger Ellison syndrome, acute pancreatitis, breast cancer, chylothorax, congenital lymphedema, diabetes mellitus, gastric paresis, hepatocellular carcinoma, non-variceal upper gastrointestinal bleeding, obestity, pancreaticoduodenectomy, prostate cancer, protein-losing enteropathy, small cell lung cancer, thyroid cancer, thyroid eye disease, vascular (arterio-venous) malformations of the gastrointestinal tract, polycystic kidney disease, Cushing's disease, GHRH-producing tumors, and other conditions resulting in abnormally elevated growth hormone, insulin, or glucagon levels in an individual in need thereof, the method comprising administering to the individual a pharmaceutical composition provided herein. In some embodiments, the pharmaceutical composition is administered orally, sublingually, subcutaneously, intravenously, intranasally, topically, transdermally, intraperitoneally, intramuscularly, intrapulmonarilly, vaginally, rectally, or intraocularly. In some embodiments, the pharmaceutical composition is administered intranasally.

### BRIEF DESCRIPTION OF THE DRAWINGS

FIG. 1 shows mean plasma concentrations of octreotide following administration of various octreotide formulations to rabbits.

FIG. 2 shows maximum plasma concentrations of octreotide following administration of various octreotide formulations to rabbits.

FIG. 3 shows the areas under the plasma concentration-time curve from time 0 to the last measurable plasma concentration following administration of various octreotide formulations to rabbits.

FIG. 4 shows mean plasma concentrations of octreotide following intranasal administration of various octreotide formulations to rabbits.

FIG. 5 shows maximum plasma concentrations of octreotide following intranasal administration of various octreotide formulations to rabbits.

FIG. 6 shows the areas under the plasma concentration-time curve from time 0 to the last measurable plasma concentration following intranasal administration of various octreotide formulations to rabbits.

FIG. 7 shows changes in degradant peaks for different formulations over 4 weeks at 40° C.

FIG. 8 shows changes in degradant peaks for different formulations over 4 weeks at 25° C.

FIG. 9 shows changes in degradant peaks for different formulations over 4 weeks at 5° C.

FIG. 10 shows the stability data for Formulation 3 in Example 4 upon storage at 40, 25, and 5 degrees Celsius over a period of 26 weeks.

FIG. 11 shows the stability data for Formulations 1 and 4 in Example 5 upon storage at 40, 25, and 5 degrees Celsius over a period of 15 weeks.

FIG. 12 shows mean plasma concentrations of lanreotide following administration of various lanreotide formulations to rabbits.

FIG. 13 shows mean plasma concentrations of octreotide following administration of the trial formulation at different dose levels to human subjects.

FIG. 14 shows growth hormone response in human subjects following administration of the trial formulation and Sandostatin IR.

### DETAILED DESCRIPTION

Provided herein are pharmaceutical compositions of therapeutic peptides that may exhibit desirable properties such as bioavailability, stability, and reduced side-effects associated with administration.

As used herein, the term “alkyl” refers to saturated or unsaturated aliphatic groups including straight-chain, branched-chain, and combinations thereof. In some embodiments, an alkyl has 1-6 carbon atoms. In some embodiments, an alkyl has 1-3 carbon atoms. In some embodiments, an alkyl has 10-16 carbon atoms. In some embodiments, an alkyl has 12-14 carbon atoms. In some embodiments, an alkyl is a saturated, straight-chain aliphatic group.

The term “hydroxyalkyl” refers to an alkyl substituted with one or more hydroxyl (—OH) moieties at any chemically feasible position. In some embodiments, the hydroxyalkyl contains one hydroxyl moiety. In some embodiments, the hydroxyalkyl contains two hydroxyl moieties. Exemplary hydroxyalkyl moieities include, without limitation, hydroxymethyl, ethan-1-ol, ethan-2-ol, 1-propan-1-ol, 1-propan-2-ol, 1-propan-3-ol, 2-propan-1-ol, and 2-propan-2-ol.

The term “peptide” refers to a polymer composed of amino acid residues, related naturally occurring structural variants, and synthetic non-naturally occurring analogs thereof. Synthetic polypeptides can be synthesized, for example, using an automated polypeptide synthesizer. A peptide comprises two or more amino acids joined to each other by peptide bonds or modified peptide bonds. Peptides may contain amino acids other than the 20 gene encoded amino acids, including non natural enantiomers of natural amino-acids. “Peptide(s)” include those modified either by natural processes, such as processing and other post-translational modifications, but also by chemical modification techniques. Such modifications are well known in the art. The same type of modification may be present in the same or varying degree at several sites in a given peptide. Also, a given peptide may contain one or more types of modifications. Modifications can occur anywhere in a peptide, including the peptide backbone, the amino acid side-chains, and the amino or carboxyl termini. Modifications include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide or thioether bond formation, demethylation, formation of covalent cross-link of cysteine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, glycosylation, lipid attachment, sulfation, gamma-carboxylation of glutamic acid residues, hydroxylation and ADP-ribosylation, selenoylation, sulfation, transfer-RNA mediated addition of amino acids to proteins, such as arginylation, and ubiquitination. Peptides may be branched or cyclic, with or without branching.

As used herein, “therapeutically effective amount” indicates an amount that results in a desired pharmacological and/or physiological effect for the condition. The effect may be prophylactic in terms of completely or partially preventing a condition or symptom thereof and/or may be therapeutic in terms of a partial or complete cure for the condition and/or adverse effect attributable to the condition.

When used with respect to methods of treatment/prevention and the use of the compositions described herein, an individual “in need thereof” may be an individual who has been diagnosed with or previously treated for the condition to be treated. With respect to prevention, the individual in need thereof may also be an individual who is at risk for a condition (e.g., a family history of the condition, life-style factors indicative of risk for the condition, etc.).

In some embodiments, the individual is a mammal, including, but not limited to, bovine, horse, feline, rabbit, canine, rodent, or primate. In some embodiments, the mammal is a primate. In some embodiments, the primate is a human, including adults, children, and premature infants. In some embodiments, the individual is a non-mammal. In some variations, the primate is a non-human primate such as chimpanzees and other apes and monkey species. In some embodiments, the mammal is a farm animal such as cattle, horses, sheep, goats, and swine; pets such as rabbits, dogs, and cats; laboratory animals including rodents, such as rats, mice, and guinea pigs; and the like. Examples of non-mammals include, but are not limited to, birds, and the like. The term “individual” does not denote a particular age or sex.

In some variations, the individual has been identified as having one or more of the conditions described herein. Identification of the conditions as described herein by a skilled physician is routine in the art and may also be suspected by the individual or others, for example, due to exhibiting the symptoms of the condition.

In some embodiments, the individual has been identified as susceptible to one or more of the conditions as described herein. The susceptibility of an individual may be based on any one or more of a number of risk factors and/or diagnostic approaches appreciated by the skilled artisan, including, but not limited to, genetic profiling, family history, medical history (e.g., appearance of related conditions), lifestyle, or habits.

As used herein, “treatment” or “treating” is an approach for obtaining a beneficial or desired result, such as a clinical result. Beneficial or desired clinical results include, but are not limited to, alleviation of a symptom and/or diminishment of the extent of a symptom and/or preventing a worsening of a symptom associated with a condition.

As used herein and in the appended claims, the singular forms “a”, “an” and “the” include plural forms, unless the context clearly dictates otherwise.

Reference to “about” a value or parameter herein includes (and describes) variations that are directed to that value or parameter per se. For example, a description referring to “about X” includes the description of “X”.

### Compositions

In one aspect, provided herein are pharmaceutical compositions, also referred to as formulations, comprising a therapeutic peptide or a pharmaceutically acceptable salt thereof, an alkylsaccharide, and a pharmaceutically acceptable excipient. In some embodiments, the alkylsaccharide consists of a C₈-C₁₆ alkyl linked by glycosidic linkage to a maltose. In some embodiments, the alkylsaccharide consists of a C₁₂ alkyl linked by glycosidic linkage to a maltose. In some embodiments, the pharmaceutically acceptable excipient is selected from the group consisting of alkyl cellulose, hydroxyalkylcellulose, hydroxyalkyl alkylcellulose and polyethylene glycol.

The pharmaceutical formulation may be a liquid, such as an aqueous liquid. In some embodiments, the formulation is a clear liquid essentially free from visible particles.

The compositions provided herein contain one or more therapeutic peptides including, without limitation, somatostatin or somatostatin polypeptide analogs, vasopressin, vasopressin polypeptide analogs, desmopressin, glucagon, corticotropin (ACTH), gonadotropin, calcitonin, C-peptide of insulin, parathyroid hormone (PTH) and cyclic or linear analogs thereof including PTH 1-31, PTH 1-34, and PTH 3-34, growth hormone (HG), human growth hormone (hGH), growth hormone releasing hormone (GHRH), oxytocin and oxytocin polypeptide analogs, corticotropin releasing hormone (CRH), gonadotropin agonist or gonadotrophin agonist polypeptide analogs, human atrial natriuretic peptide (ANP), human thyroxine releasing hormone (TRH), follicle stimulating hormone (FSH), prolactin, insulin, insulin like growth factor-I (IGF-I) somatomedin-C(SM-C), calcitonin, leptin and the leptin derived short peptide OB-3, melatonin, GLP-1 or Glucagon-like peptide-1, GiP, pituitary adenylate cyclase activating peptide, GM-1 ganglioside, nerve growth factor (NGF), nafarelin, D-tryp6)-LHRH, FGF, VEGF antagonists, leuprolide, interferon (e.g., α, β, γ) low molecular weight heparin, PYY, LHRH antagonists, Keratinocyte Growth Factor (KGF), Glial-Derived Neurotrophic Factor (GDNF), testosterone, ghrelin, ghrelin antagonists, vasoactive intestinal peptide, and apelin or analogs thereof (e.g., truncated forms).

The therapeutic peptide may contain between about 2 and about 50 amino acids, such as between about 5 and about 50 amino acids, between about 10 and about 50 amino acids, between about 15 and about 50 amino acids, between about 20 and about 50 amino acids, between about 25 and about 50 amino acids, between about 30 and about 50 amino acids, between about 35 and about 50 amino acids, between about 40 and about 50 amino acids, between about 45 and about 50 amino acids, between about 2 and about 45 amino acids, between about 5 and about 45 amino acids, between about 10 and about 45 amino acids, between about 15 and about 45 amino acids, between about 20 and about 45 amino acids, between about 25 and about 45 amino acids, between about 30 and about 45 amino acids, between about 35 and about 45 amino acids, between about 40 and about 45 amino acids, between about 2 and about 40 amino acids, between about 5 and about 40 amino acids, between about 10 and about 40 amino acids, between about 15 and about 40 amino acids, between about 20 and about 40 amino acids, between about 25 and about 40 amino acids, between about 30 and about 40 amino acids, between about 35 and about 40 amino acids, between about 2 and about 35 amino acids, between about 5 and about 35 amino acids, between about 10 and about 35 amino acids, between about 15 and about 35 amino acids, between about 20 and about 35 amino acids, between about 25 and about 35 amino acids, between about 30 and about 35 amino acids, between about 2 and about 30 amino acids, between about 5 and about 30 amino acids, between about 10 and about 30 amino acids, between about 15 and about 30 amino acids, between about 20 and about 30 amino acids, between about 25 and about 30 amino acids, between about 2 and about 25 amino acids, between about 5 and about 25 amino acids, between about 10 and about 25 amino acids, between about 15 and about 25 amino acids, between about 20 and about 25 amino acids, between about 2 and about 20 amino acids, between about 5 and about 20 amino acids, between about 10 and about 20 amino acids, between about 15 and about 20 amino acids, between about 2 and about 15 amino acids, between about 5 and about 15 amino acids, between about 10 and about 15 amino acids, between about 2 and about 10 amino acids, between about 5 and about 10 amino acids, or between about 2 and about 5 amino acids. In some embodiments, the therapeutic peptide contains at least about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, or about 45 amino acids. In some embodiments, the therapeutic peptide contains less than about 50, about 49, about 48, about 47, about 46, about 45, about 44, about 43, about 42, about 41, about 40, about 39, about 38, about 37, about 36, about 35, about 34, about 33, about 32, about 31, about 30, about 29, about 28, about 27, about 26, about 25, about 24, about 23, about 22, about 21, about 20, about 19, about 18, about 17, about 16, about 15, about 14, about 13, about 12, about 11, about 10, about 9, about 8, about 7, about 6, or about 5 amino acids. In some embodiments, the therapeutic peptide is a cyclic peptide. Cyclic peptides provided herein may be branched or unbranched.

Therapeutic peptides described herein, including cyclic peptides, may be readily synthesized by any known procedure for the formation of a peptide linkage between amino acids. Such procedures include, for example, any solution phase procedure permitting a condensation between the free alpha amino group of an amino acid residue having its carboxyl group or other reactive groups protected and the free primary carboxyl group of another amino acid residue having its amino group or other reactive groups protected.

The process for synthesizing the peptides, such as cyclic peptides, may be carried out by a procedure whereby each amino acid in the desired sequence is added one at a time in succession to another amino acid residue or by a procedure whereby peptide fragments with the desired amino acid sequence are first synthesized and then condensed to provide the desired peptide. The resulting peptide is then cyclized to yield a cyclic peptide. A cyclic peptide can be obtained by inducing the formation of a covalent bond between an amino group at the N-terminus of the peptide, if provided, and a carboxyl group at the C-terminus, if provided. A cyclic peptide can also be obtained by forming a covalent bond between a terminal reactive group and a reactive amino acid side chain moiety, or between two reactive amino acid side chain moieties (such as cysteine). The means by which a given peptide is made cyclic may be determined by the reactive groups present in the peptide and the desired characteristic of the peptide.

In some embodiments, the therapeutic peptide is somatostatin or an analog thereof or a pharmaceutically acceptable salt of the foregoing. Somatostatin is a peptide hormone that regulates the endocrine system and affects neurotransmission and cell proliferation via interaction with G-protein coupled receptors that results in the inhibition of release of several secondary hormones, including growth hormone and thyroid-stimulating hormone (TSH) in the anterior pituitary; gastrin, cholecystokinin, motilin, glucagon, secretin, pancreatic polypeptide, thyroid stimulating hormone (TSH), gastric inhibitory peptide (GIP), enteroglucagon, and vasoactive intestinal peptide (VIP) in the gastrointestinal system; and insulin and glucagon in the pancreas. Somatostatin is a tetradecapeptide having the following structure.

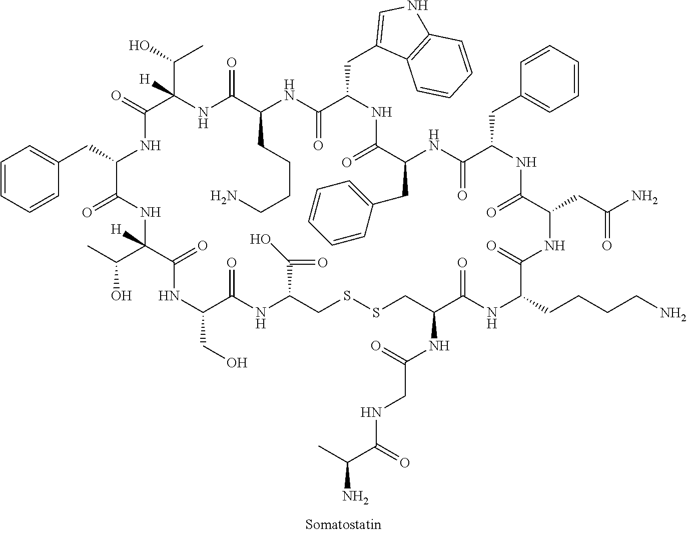 [SMILES: C[C@H](N)C(=O)NCC(=O)N[C@H]1CSSC[C@@H](C(=O)O)NC(=O)[C@H](CO)NC(=O)[C@H]([C@@H](C)O)NC(=O)[C@H](Cc2ccccc2)NC(=O)[C@H]([C@@H](C)O)NC(=O)[C@H](CCCCN)NC(=O)[C@H](Cc2c[nH]c3ccccc23)NC(=O)[C@H](Cc2ccccc2)NC(=O)[C@H](Cc2ccccc2)NC(=O)[C@H](CC(N)=O)NC(=O)[C@H](CCCCN)NC1=O]

In some embodiments, the composition contains an analog of somatostatin. In some embodiments, the somatostatin analog is a straight-chain or cyclic polypeptide having a structure based on that of the naturally occurring somatostatin, wherein one or more amino acid units have been omitted and/or replaced by one or more other amino acid radical(s) and/or wherein one or more functional groups have been replaced by one or more other functional groups and/or one or more groups have been replaced by one or several other isosteric groups. In some embodiments, the somatostatin analog exhibits a somatostatin-related activity, e.g., it binds to at least one of the five somatostatin receptors (SSTR). Somatostatin analogs that can be present in the compositions provided herein include, without limitation, octreotide, lanreotide, and pasireotide, and pharmaceutically acceptable salts of any of the foregoing.

Octreotide (D-phenylalanyl-L-cysteinyl-L-phenylalanyl-D-tryptophyl-L-lysyl-L-threonyl-N-[2-hydroxy-1-(hydroxymethyl)propyl]-L-Cysteinamide, cyclic (2→7)-disulfide) is a synthetic octapeptide mimic of the natural peptide somatostatin. It inhibits the secretion of certain hormones, including gastrin, cholecystokinin, glucagon, growth hormone, insulin, secretin, pancreatic polypeptide, thyroid stimulating hormone (TSH), and vasoactive intestinal peptide (VIP). Octreotide has the following structure:

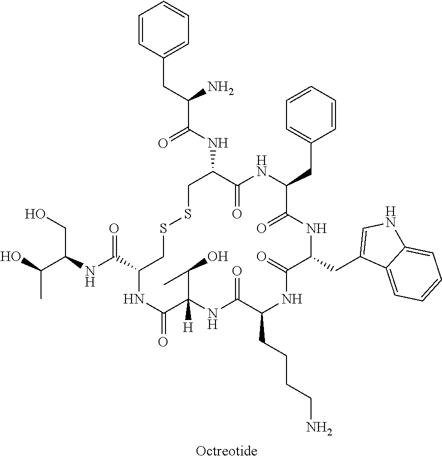 [SMILES: C[C@@H](O)[C@@H]1NC(=O)[C@H](CCCCN)NC(=O)[C@@H](Cc2c[nH]c3ccccc23)NC(=O)[C@H](Cc2ccccc2)NC(=O)[C@@H](NC(=O)[C@H](N)Cc2ccccc2)CSSC[C@@H](C(=O)N[C@H](CO)[C@@H](C)O)NC1=O]

Lanreotide ([cyclo S—S]-3-(2-naphthyl)-D-alanyl-L-cysteinyl-L-tyrosyl-D-tryptophyl-L-lysyl-L-valyl-L-cysteinyl-L-threoninamide) is also a synthetic octapeptide mimic of the natural peptide somatostatin. Lanreotide has the following structure:

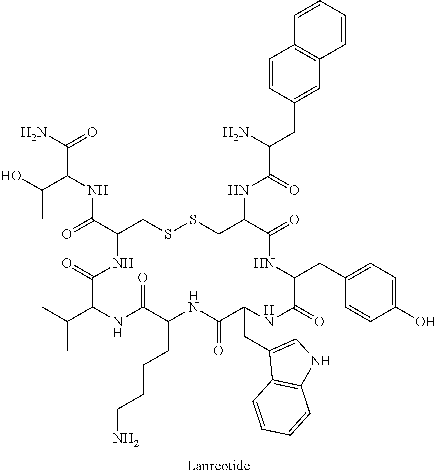 [SMILES: CC(C)C1NC(=O)C(CCCCN)NC(=O)C(Cc2c[nH]c3ccccc23)NC(=O)C(Cc2ccc(O)cc2)NC(=O)C(NC(=O)C(N)Cc2ccc3ccccc3c2)CSSCC(C(=O)NC(C(N)=O)C(C)O)NC1=O]

Pasireotide ((2-Aminoethyl) carbamic acid (2R,5 S, 8 S,11S,14R,17S,19aS)-11-(4-aminobutyl)-5-benzyl-8-(4-benzyloxybenzyl)-14-(1H-indol-3 ylmethyl)-4,7,10,13,16,19-hexaoxo-17-phenyloctadecahydro-3a, 6,9,12,15,18 hexaazacyclopentacyclooctadecen-2-yl ester) is a cyclohexapeptide mimic of the natural peptide somatostatin. Pasireotide has the following structure:

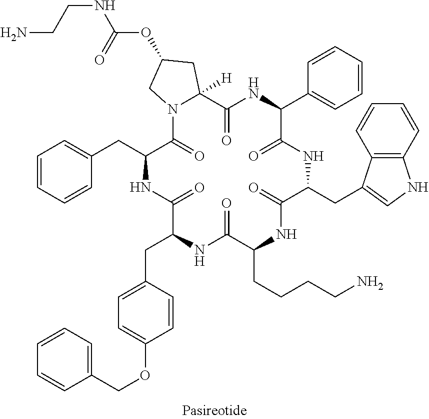 [SMILES: NCCCC[C@@H]1NC(=O)[C@@H](Cc2c[nH]c3ccccc23)NC(=O)[C@H](c2ccccc2)NC(=O)[C@@H]2C[C@@H](OC(=O)NCCN)CN2C(=O)[C@H](Cc2ccccc2)NC(=O)[C@H](Cc2ccc(OCc3ccccc3)cc2)NC1=O]

The therapeutic peptide of the compositions described herein can be provided in the form of a pharmaceutically acceptable salt. Pharmaceutically acceptable salts include acid addition salts formed with inorganic acids or organic acids and base addition salts formed with bases. Inorganic acids suitable for forming pharmaceutically acceptable salts include, without limitation, hydrohalide acids (e.g., hydrochloric acid, hydrobromic acid, hydriodic acid), sulfuric acid, nitric acid, phosphoric acid, and the like. Organic acids suitable for forming pharmaceutically acceptable salts include, without limitation, acetic acid, trifluoroacetic acid, propionic acid, hexanoic acid, cyclopentanepropionic acid, glycolic acid, oxalic acid, pyruvic acid, lactic acid, malonic acid, succinic acid, malic acid, maleic acid, fumaric acid, tartaric acid, citric acid, palmitic acid, benzoic acid, 3-(4-hydroxybenzoyl) benzoic acid, cinnamic acid, mandelic acid, alkylsulfonic acids (e.g., methanesulfonic acid, ethanesulfonic acid, 1,2-ethane-disulfonic acid, 2-hydroxyethanesulfonic acid, etc.), arylsulfonic acids (e.g., benzenesulfonic acid, 4-chlorobenzenesulfonic acid, 2-naphthalenesulfonic acid, 4-toluenesulfonic acid, camphorsulfonic acid, etc.), 4-methylbicyclo(2.2.2)-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, aspartic acid and the like. Pharmaceutically acceptable salts formed with bases include, without limitation, aluminum, ammonium, calcium, cupric, cuprous, ferric, ferrous, lithium, magnesium, manganese (-ic and -ous), potassium, sodium, zinc and the like salts.

In some embodiments, the therapeutic peptide is octreotide acetate. In some embodiments, the therapeutic peptide is lanreotide acetate. In some embodiments, the therapeutic peptide is pasireotide diaspartate. In some embodiments, the pharmaceutical composition contains two or more therapeutic peptides, such as two or more somatostatin analogs. Particular combinations of therapeutic peptides include, without limitation, octreotide and lanreotide, octreotide and pasireotide, and lanreotide and pasireotide. In some embodiments, the pharmaceutical composition contains a single therapeutic peptide.

In some embodiments, the pharmaceutical composition contains between about 0.01% (w/w) and about 10% (w/w) of the therapeutic peptide or a pharmaceutically acceptable salt thereof, such as between about 0.05% (w/w) and about 10% (w/w), between about 0.1% (w/w) and about 10% (w/w), between about 0.5% (w/w) and about 10% (w/w), between about 1% (w/w) and about 10% (w/w), between about 0.01% (w/w) and about 7% (w/w), between about 0.05% (w/w) and about 7% (w/w), between about 0.1% (w/w) and about 7% (w/w), between about 0.5% (w/w) and about 7% (w/w), between about 1% (w/w) and about 7% (w/w), between about 0.01% (w/w) and about 5% (w/w), between about 0.05% (w/w) and about 5% (w/w), between about 0.1% (w/w) and about 5% (w/w), between about 0.5% (w/w) and about 5% (w/w), between about 1% (w/w) and about 5% (w/w), between about 0.01% (w/w) and about 3% (w/w), between about 0.05% (w/w) and about 3% (w/w), between about 0.1% (w/w) and about 3% (w/w), between about 0.5% (w/w) and about 3% (w/w), or between about 1% (w/w) and about 3% (w/w). In some embodiments, the pharmaceutical composition contains at least about 0.01%, about 0.05%, about 0.1%, about 0.2%, about 0.3%, about 0.4%, about 0.5%, about 0.6%, about 0.7%, about 0.8%, about 0.9%, or about 1% of a therapeutic peptide or a pharmaceutically acceptable salt thereof. In some embodiments, the pharmaceutical composition contains less than about 10%, about 9%, about 8%, about 7%, about 6%, about 5%, about 4%, or about 3% of the therapeutic peptide or a pharmaceutically acceptable salt thereof.

In some embodiments, the pharmaceutical composition provided herein contains an alkylsaccharide. An alkylsaccharide may be any saccharide joined by a linkage to any alkyl. In some embodiments, the alkylsaccharide is nonionic, as well as nontoxic and considered Generally Recognized As Safe for food applications, sometimes referred to as a GRAS substance. Alkylsaccharides may be synthesized by known procedures, such as chemically or enzymatically.

Examples of alkylglycosides can be used in the pharmaceutical compositions provided herein include: alkylglycosides, such as octyl-, nonyl-, decyl-, undecyl-, dodecyl-, tridecyl-, tetradecyl, pentadecyl-, hexadecyl-, heptadecyl-, and octadecyl-D-maltoside, -glucoside or -sucroside (i.e., sucrose ester); alkyl thiomaltosides, such as heptyl-, octyl-, dodecyl-, tridecyl-, and tetradecyl-β-D-thiomaltoside; alkyl thioglucosides, such as heptyl- or octyl 1-thio β- or β-D-glucopyranoside; alkyl thiosucroses; alkyl maltotriosides; long chain aliphatic carbonic acid amides of sucrose amino-alkyl ethers; derivatives of palatinose and isomaltamine linked by amide linkage to an alkyl chain; derivatives of isomaltamine linked by urea to an alkyl chain; long chain aliphatic carbonic acid ureides of sucrose amino-alkyl ethers; and long chain aliphatic carbonic acid amides of sucrose amino-alkyl ethers.

Particular glycosides include maltose, sucrose, and glucose linked by glycosidic or ester linkage to an alkyl chain of 9, 10, 12, 13 or 14 carbon atoms, e.g., nonyl-, decyl-, dodecyl- and tetradecyl sucroside, glucoside, and maltoside.

In some embodiments, the saccharide portion of the alkylsaccharide is a monosaccharide. Exemplary monosaccharides include, without limitation, allose, altrose, glucose, mannose, gulose, idose, galactose, talose, psicose, fructose, sorbose, tagatose, arabinose, lyxose, ribose, xylose, ribulose, and xylulose. In some embodiments, the saccharide portion of the alkylsaccharide is a disaccharide. Exemplary disaccharides include, without limitation, sucrose, lactulose, lactose, maltose, trehalose, cellobiose, chitobiose, kojibiose, nigerose, isomaltose, sophorose, laminaribiose, gentiobiose, turanose, maltulose, palatinose, gentiobiulose, mannobiose, melibiose, malibiulose, rutinose, rutinulose, and xylobiose. In some embodiments, the saccharide portion of the alkylsaccharide is a polysaccharide. In a particular embodiment, the sacchride in the alkylsaccharide provided herein is maltose. In another particular embodiment, the sacchride in the alkylsaccharide provided herein is sucrose. In yet another particular embodiment, the sacchride in the alkylsaccharide provided herein is glucose. In any of the alkylsaccharides provided herein, one or more saccharide moiety may be in the D stereochemical configuration. In any of the alkylsaccharides provided herein, one or more saccharide moiety may be in the L stereochemical configuration. In some embodiments, the alkylglycoside is a β anomer. In some embodiments, the alkylglycoside is an a anomer. In some embodiments, the alkylglycoside is dodecyl β-D-maltoside.

In some embodiments, the alkyl portion of the alkylsaccharide contains between about 4 and about 30 carbon atoms, such as between about 4 and about 28 carbon atoms, between about 4 and about 26 carbon atoms, between about 4 and about 24 carbon atoms, between about 4 and about 22 carbon atoms, between about 4 and about 20 carbon atoms, between about 4 and about 18 carbon atoms, between about 4 and about 16 carbon atoms, between about 4 and about 14 carbon atoms, between about 4 and about 12 carbon atoms, between about 4 and about 10 carbon atoms, between about 4 and about 8 carbon atoms, between about 4 and about 6 carbon atoms, between about 8 and about 30 carbon atoms, between about 8 and about 28 carbon atoms, between about 8 and about 26 carbon atoms, between about 8 and about 24 carbon atoms, between about 8 and about 22 carbon atoms, between about 8 and about 20 carbon atoms, between about 8 and about 18 carbon atoms, between about 8 and about 16 carbon atoms, between about 8 and about 14 carbon atoms, between about 8 and about 12 carbon atoms, between about 8 and about 10 carbon atoms, between about 12 and about 30 carbon atoms, between about 12 and about 28 carbon atoms, between about 12 and about 26 carbon atoms, between about 12 and about 24 carbon atoms, between about 12 and about 22 carbon atoms, between about 12 and about 20 carbon atoms, between about 12 and about 18 carbon atoms, between about 12 and about 16 carbon atoms, between about 12 and about 14 carbon atoms, between about 16 and about 30 carbon atoms, between about 16 and about 28 carbon atoms, between about 16 and about 26 carbon atoms, between about 16 and about 24 carbon atoms, between about 16 and about 22 carbon atoms, between about 16 and about 20 carbon atoms, between about 16 and about 18 carbon atoms, between about 20 and about 30 carbon atoms, between about 20 and about 28 carbon atoms, between about 20 and about 26 carbon atoms, between about 20 and about 24 carbon atoms, between about 20 and about 22 carbon atoms, between about 24 and about 30 carbon atoms, between about 24 and about 28 carbon atoms, between about 24 and about 26 carbon atoms, or between about 26 and about 30 carbon atoms. In some embodiments, the alkyl chain of the alkylsaccharide contains at least about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 carbon atoms. In some embodiments, the alkyl chain of the alkylsaccharide contains less than about 30, about 29, about 28, about 27, about 26, about 25, about 24, about 23, about 22, about 21, about 20, about 19, about 18, about 17, about 16, about 15, about 14, about 13, about 12, about 11, about 10, about 9, about 8, about 7, about 6, about 5, about 4, or about 3 carbon atoms. In some embodiments, the alkyl chain of the alkylsaccharide contains between about 12 and about 14 carbon atoms.

The linkage between the alkyl group and the saccharide may be, without limitation, a glycosidic, thioglycosidic, amide, ureide, or ester linkage.

In some embodiments, the alkylsacchride contains maltose, linked by glycosidic linkage to an alkyl chain of about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 carbon atoms. In some embodiments, the alkylsacchride contains sucrose, linked by glycosidic linkage to an alkyl chain of about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 carbon atoms. In some embodiments, the alkylsacchride contains glucose, linked by glycosidic linkage to an alkyl chain of about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 carbon atoms.

Particular examples of alkylsaccharides include, without limitation, sucrose cocoate, n-Dodecyl-4-O-α-D-glucopyranosyl-β-D-glucopyranoside (also known as DDM), n-tetradecyl-4-O-α-D-glucopyranosyl-β-D-glucopyranoside, sucrose laurate, sucrose myristate, sucrose palmitate and mixtures thereof. In some embodiments, the alkylsaccharide is n-Dodecyl-4-O-α-D-glucopyranosyl-β-D-glucopyranoside (DDM).

In some embodiments, the alkylglycoside is in the β anomer form with less than about 2% of the α anomer form, less than about 1.5% of the α anomer form, or less than about 1% of the α anomer form. In some embodiments, the alkylgycoside contains greater than 98% β anomer, greater than 99% β anomer, greater than 99.5% β anomer, or greater than 99.9% β anomer.

In some embodiments, the alkylglycoside is in the α anomer form with less than about 2% of the β anomer form, less than about 1.5% of the β anomer form, or less than about 1% of the β anomer form. In some embodiments, the alkylgycoside contains greater than 98% a anomer, greater than 99% α anomer, greater than 99.5% α anomer, or greater than 99.9% a anomer.

In some embodiments, the pharmaceutical composition contains between about 0.01% (w/w) and about 5% (w/w) of the alkylsaccharide, such as between about 0.02% (w/w) and about 5% (w/w), between about 0.05% (w/w) and about 5% (w/w), between about 0.1% (w/w) and about 5% (w/w), between about 0.2% (w/w) and about 5% (w/w), between about 0.5% (w/w) and about 5% (w/w), between about 1% (w/w) and about 5% (w/w), between about 2% (w/w) and about 5% (w/w), between 0.02% (w/w) and about 5% (w/w), between about 0.05% (w/w) and about 5% (w/w), between about 0.1% (w/w) and about 5% (w/w), between about 0.2% (w/w) and about 5% (w/w), between about 0.5% (w/w) and about 5% (w/w), between about 1% (w/w) and about 5% (w/w), between about 2% (w/w) and about 5% (w/w), between 0.01% (w/w) and about 2.5% (w/w), between about 0.02% (w/w) and about 2.5% (w/w), between about 0.05% (w/w) and about 2.5% (w/w), between about 0.1% (w/w) and about 2.5% (w/w), between about 0.2% (w/w) and about 2.5% (w/w), between about 0.5% (w/w) and about 2.5% (w/w), between about 1% (w/w) and about 2.5% (w/w), between about 2% (w/w) and about 2.5% (w/w), between 0.01% (w/w) and about 1% (w/w), between about 0.02% (w/w) and about 1% (w/w), between about 0.05% (w/w) and about 1% (w/w), between about 0.1% (w/w) and about 1% (w/w), between about 0.2% (w/w) and about 1% (w/w), or between about 0.5% (w/w) and about 1% (w/w). In some embodiments, the pharmaceutical composition contains at least about 0.01%, about 0.05%, about 0.1%, about 0.2%, about 0.3%, about 0.4%, about 0.5%, about 0.6%, about 0.7%, about 0.8%, about 0.9%, or about 1% of the alkylsaccharide. In some embodiments, the pharmaceutical composition contains less than about 5%, about 4%, about 3%, about 2%, or about 1% of the alkylsaccharide.

The pharmaceutical composition contains a pharmaceutically acceptable excipient, which may be selected from the group consisting of polyethylene glycol of average molecular weight less than about 360 Dalton, alkylcellulose, hydroxyalkyl cellulose, and hydroxyalkyl alkylcellulose. In some embodiments, the excipient is water-soluble. In some embodiments, the excipient is non-ionic.

An alkylcellulose provided herein may be a cellulose polymer in which one or more hydroxyl groups of the cellulose backbone has been etherified to produce an alkyl ether. Etherification of cellulose may be achieved by any method known in the art. Etherification of the cellulose may be total or partial. In some embodiments, at least about 1, about 2, about 5, about 10, about 20, about 30, about 40, about 50, about 60, about 70, about 80, about 90, or about 95% of the hydroxyl groups of the cellulose backbone are etherified. The degree of etherification may vary across an alkylcellulose batch and between different batches. In some embodiments, the alkylcellulose is methylcellulose. In some embodiments, the alkylcellulose is ethylcellulose. In some embodiments, the etherification of the cellulose is partial, and one or more of the remaining hydroxyl groups of the cellulose backbone is functionalized to contain an additional chemical moiety (e.g., an ester, a sulphate, a nitrate, a carboxyalkyl). In other embodiments, the etherification of the cellulose is partial, and the remaining hydroxyl groups of the cellulose backbone are free or substantially free of other functional groups.

A hydroxyalkyl cellulose provided herein may be a cellulose polymer in which one or more hydroxyl groups of the cellulose backbone has been etherified to produce a hydroxyalkyl ether. Etherification of cellulose may be achieved by any method known in the art. Etherification of the cellulose may be total or partial. In some embodiments, at least about 1, about 2, about 5, about 10, about 20, about 30, about 40, about 50, about 60, about 70, about 80, about 90, or about 95% of the hydroxyl groups of the cellulose backbone are etherified. The degree of etherification may vary across a hydroxyalkylcellulose batch and between different batches. In some embodiments, the hydroxyalkylcellulose is hydroxymethylcellulose. In some embodiments, the hydroxyalkylcellulose is hydroxyethylcellulose. In some embodiments, hydroxyalkylcellulose is hydroxypropylcellulose. In some embodiments, the etherification of the cellulose is partial, and one or more of the remaining hydroxyl groups of the cellulose backbone is functionalized to contain an additional chemical moiety (e.g., an ester, a sulphate, a nitrate, a carboxyalkyl). In other embodiments, the etherification of the cellulose is partial, and the remaining hydroxyl groups of the cellulose backbone are free or substantially free of other functional groups.

A hydroxyalkyl alkylcellulose provided herein may be a cellulose polymer in which one or more hydroxyl groups of the cellulose backbone has been etherified to produce a hydroxyalkyl ether and one or more hydroxyl groups of the cellulose backbone has been etherified to produce an alkyl ether. Etherification of cellulose may be achieved by any method known in the art. Etherification of the cellulose may be total or partial. In some embodiments, at least about 1, about 2, about 5, about 10, about 20, about 30, about 40, about 50, about 60, about 70, about 80, about 90, or about 95% of the hydroxyl groups of the cellulose backbone are etherified with a hydroxyalkyl ether. In some embodiments, at least about 1, about 2, about 5, about 10, about 20, about 30, about 40, about 50, about 60, about 70, about 80, about 90, or about 95% of the hydroxyl groups of the cellulose backbone are etherified with an alkyl ether. The degree of etherification may vary across a hydroxyalkyl alkylcellulose batch and between different batches. In some embodiments, the hydroxyalkyl alkylcellulose is hydroxymethyl methylcellulose. In some embodiments, the hydroxyalkylcellulose is hydroxyethyl methylcellulose. In some embodiments, the hydroxyalkylcellulose is hydroxypropyl methylcellulose. In some embodiments, the etherification of the cellulose is partial, and one or more of the remaining hydroxyl groups of the cellulose backbone is functionalized to contain an additional chemical moiety (e.g., an ester, a sulphate, a nitrate, a carboxyalkyl). In other embodiments, the etherification of the cellulose is partial, and the remaining hydroxyl groups of the cellulose backbone are free or substantially free of other functional groups.

In some embodiments, the pharmaceutically acceptable excipient is hydroxyethyl cellulose (HEC), hydroxylpropyl cellulose (HPC), hydroxyethyl methyl cellulose (HEMC), hydroxypropyl methyl cellulose (HPMC), or ethyl hydroxyethyl cellulose.

In some embodiments, the pharmaceutically acceptable excipient is METHOCEL™ (Dow Chemical Company). In a particular embodiment, the pharmaceutically acceptable excipient is METHOCEL™ E4M.

In some embodiments, the pharmaceutically acceptable excipient is a polyethylene glycol. In some embodiments, the average molecular weight of the polyethylene glycol is less than 360 Dalton. In some embodiments, the polyethylene glycol has an average molecular weight of between about 50 Dalton and 350 Dalton, such as between about 50 Dalton and about 300 Dalton, between about 50 Dalton and about 250 Dalton, between about 50 Dalton and about 200 Dalton, between about 50 Dalton and about 150 Dalton, between about 50 Dalton and about 100 Dalton, between about 100 Dalton and about 350 Dalton, between about 100 Dalton and about 300 Dalton, between about 100 Dalton and about 250 Dalton, between about 100 Dalton and about 200 Dalton, between about 100 Dalton and about 150 Dalton, between about 150 Dalton and about 350 Dalton, between about 150 Dalton and about 300 Dalton, between about 150 Dalton and about 250 Dalton, between about 150 Dalton and about 200 Dalton, between about 200 Dalton and about 350 Dalton, between about 200 Dalton and about 300 Dalton, between about 200 Dalton and about 250 Dalton, between about 250 Dalton and about 350 Dalton, between about 250 Dalton and about 300 Dalton, between about 300 Dalton and about 350 Dalton, or between about 190 Dalton and about 210 Dalton. In some embodiments, the polyethylene glycol has an average molecular weight of at least about 50 Dalton, about 60 Dalton, about 70 Dalton, about 80 Dalton, about 90 Dalton, about 100 Dalton, about 110 Dalton, about 120 Dalton, about 130 Dalton, about 140 Dalton, about 150 Dalton, about 160 Dalton, about 170 Dalton, about 180 Dalton, about 190 Dalton, about 200 Dalton, about 210 Dalton, about 220 Dalton, about 230 Dalton, about 240 Dalton, about 250 Dalton, about 260 Dalton, about 270 Dalton, about 280 Dalton, about 290 Dalton, about 300 Dalton, about 310 Dalton, about 320 Dalton, about 330 Dalton, or about 340 Dalton. In some embodiments, the polyethylene glycol has an average molecular weight of less than about 50 Dalton, about 60 Dalton, about 70 Dalton, about 80 Dalton, about 90 Dalton, about 100 Dalton, about 110 Dalton, about 120 Dalton, about 130 Dalton, about 140 Dalton, about 150 Dalton, about 160 Dalton, about 170 Dalton, about 180 Dalton, about 190 Dalton, about 200 Dalton, about 210 Dalton, about 220 Dalton, about 230 Dalton, about 240 Dalton, about 250 Dalton, about 260 Dalton, about 270 Dalton, about 280 Dalton, about 290 Dalton, about 300 Dalton, about 310 Dalton, about 320 Dalton, about 330 Dalton, about 340 Dalton, about 350 Dalton, or about 360 Dalton.

In some embodiments, the pharmaceutically acceptable excipient is PEG 200.

In some embodiments, the pharmaceutical composition contains between about 0.01% (w/w) and about 5% (w/w) of the pharmaceutically acceptable excipient, such as between about 0.02% (w/w) and about 5% (w/w), between about 0.05% (w/w) and about 5% (w/w), between about 0.1% (w/w) and about 5% (w/w), between about 0.2% (w/w) and about 5% (w/w), between about 0.5% (w/w) and about 5% (w/w), between about 1% (w/w) and about 5% (w/w), between about 2% (w/w) and about 5% (w/w), between 0.02% (w/w) and about 5% (w/w), between about 0.05% (w/w) and about 5% (w/w), between about 0.1% (w/w) and about 5% (w/w), between about 0.2% (w/w) and about 5% (w/w), between about 0.5% (w/w) and about 5% (w/w), between about 1% (w/w) and about 5% (w/w), between about 2% (w/w) and about 5% (w/w), between 0.01% (w/w) and about 2.5% (w/w), between about 0.02% (w/w) and about 2.5% (w/w), between about 0.05% (w/w) and about 2.5% (w/w), between about 0.1% (w/w) and about 2.5% (w/w), between about 0.2% (w/w) and about 2.5% (w/w), between about 0.5% (w/w) and about 2.5% (w/w), between about 1% (w/w) and about 2.5% (w/w), between about 2% (w/w) and about 2.5% (w/w), between 0.01% (w/w) and about 1% (w/w), between about 0.02% (w/w) and about 1% (w/w), between about 0.05% (w/w) and about 1% (w/w), between about 0.1% (w/w) and about 1% (w/w), between about 0.2% (w/w) and about 1% (w/w), or between about 0.5% (w/w) and about 1% (w/w). In some embodiments, the pharmaceutical composition contains at least about 0.01%, about 0.05%, about 0.1%, about 0.2%, about 0.3%, about 0.4%, about 0.5%, about 0.6%, about 0.7%, about 0.8%, about 0.9%, or about 1% of the pharmaceutically acceptable excipient. In some embodiments, the pharmaceutical composition contains less than about 5%, about 4%, about 3%, about 2%, or about 1% of the pharmaceutically acceptable excipient.

In some embodiments, the pharmaceutical composition includes a tonicity agent. A tonicity agent may be used to adjust the osmolarity of the composition. Suitable tonicity agents include, but are not limited to, glycerin, lactose, mannitol, dextrose, sodium chloride, sodium sulfate, sorbitol and the like.

In some embodiments, the tonicity agent is mannitol. In some embodiments, the tonicity agent is an acetate. In some embodiments, the tonicity agent is a polyol. In some embodiments, the tonicity agent is NaCl.

In some embodiments, the pharmaceutical composition contains between about 0.01% (w/w) and about 10% (w/w) of the tonicity agent, such as between about 0.05% (w/w) and about 10% (w/w), between about 0.1% (w/w) and about 10% (w/w), between about 0.5% (w/w) and about 10% (w/w), between about 1% (w/w) and about 10% (w/w), between about 0.01% (w/w) and about 10% (w/w), between about 0.05% (w/w) and about 10% (w/w), between about 0.1% (w/w) and about 10% (w/w), between about 0.5% (w/w) and about 10% (w/w), between about 1% (w/w) and about 10% (w/w), between about 0.01% (w/w) and about 9% (w/w), between about 0.05% (w/w) and about 9% (w/w), between about 0.1% (w/w) and about 9% (w/w), between about 0.5% (w/w) and about 9% (w/w), between about 1% (w/w) and about 9% (w/w), between about 0.01% (w/w) and about 8% (w/w), between about 0.05% (w/w) and about 8% (w/w), between about 0.1% (w/w) and about 8% (w/w), between about 0.5% (w/w) and about 8% (w/w), between about 1% (w/w) and about 8% (w/w), between about 0.01% (w/w) and about 7% (w/w), between about 0.05% (w/w) and about 7% (w/w), between about 0.1% (w/w) and about 7% (w/w), between about 0.5% (w/w) and about 7% (w/w), between about 1% (w/w) and about 7% (w/w), between about 0.01% (w/w) and about 6% (w/w), between about 0.05% (w/w) and about 6% (w/w), between about 0.1% (w/w) and about 6% (w/w), between about 0.5% (w/w) and about 6% (w/w), between about 1% (w/w) and about 6% (w/w), between about 0.01% (w/w) and about 5% (w/w), between about 0.05% (w/w) and about 5% (w/w), between about 0.1% (w/w) and about 5% (w/w), between about 0.5% (w/w) and about 5% (w/w), between about 1% (w/w) and about 5% (w/w), between about 0.01% (w/w) and about 4% (w/w), between about 0.05% (w/w) and about 4% (w/w), between about 0.1% (w/w) and about 4% (w/w), between about 0.5% (w/w) and about 4% (w/w), between about 1% (w/w) and about 4% (w/w), between about 0.01% (w/w) and about 3% (w/w), between about 0.05% (w/w) and about 3% (w/w), between about 0.1% (w/w) and about 3% (w/w), between about 0.5% (w/w) and about 3% (w/w), or between about 1% (w/w) and about 3% (w/w). In some embodiments, the pharmaceutical composition contains at least about 0.01%, about 0.05%, about 0.1%, about 0.2%, about 0.3%, about 0.4%, about 0.5%, about 0.6%, about 0.7%, about 0.8%, about 0.9%, or about 1% of the tonicity agent. In some embodiments, the pharmaceutical composition contains less than about 10%, about 9%, about 8%, about 7%, about 6%, about 5%, about 4%, about 3%, about 2%, or about 1% of the tonicity agent.

In some embodiments, the pharmaceutical composition contains a buffering agent. Suitable buffering agents include, without limitation, citrate buffers, maleate buffers, borate buffers, acetate buffers, lactate buffers, and combinations thereof. In some embodiments, the buffering agent is an acetate buffer. In some embodiments, the buffer also serves as a tonicity agent.

In some embodiments, the pH of the composition is between about 3 and about 8, such as between about 3.5 and about 8, between about 4 and about 8, between about 4.5 and about 8, between about 5 and about 8, between about 5.5 and about 8, between about 6 and about 8, between about 3 and about 7, between about 3.5 and about 7, between about 4 and about 7, between about 4.5 and about 7, between about 5 and about 7, between about 5.5 and about 7, between about 6 and about 7, between about 3 and about 6, between about 3.5 and about 6, between about 4 and about 6, between about 4.5 and about 6, between about 5 and about 6, between about 3 and about 5, between about 3.5 and about 5, or between about 4 and about 5. In some embodiments, the pH of the composition is at least about 3, about 3.5, about 4, about 4.5, about 5, about 5.5, about 6, about 6.5, about 7, or about 7.5. In some embodiments, the pH of the composition is less than about 8, about 7.5, about 7, about 6.5, about 6, about 5.5, about 5, about 4.5, about 4, or about 3.5.

In some embodiments, the pharmaceutical composition contains a mucoadhesive.

In some embodiments, the pharmaceutical composition contains a viscosity enhancer.

In some embodiments, the pharmaceutical composition contains a therapeutic peptide or a pharmaceutically acceptable salt thereof; an alkylsaccharide, wherein the alkylsaccharide consists of an alkyl chain length from 10 to 16 carbon atoms (e.g., 12 carbon atoms) linked by glycosidic linkage to a maltose; and a pharmaceutically acceptable excipient selected from the group consisting of alkylcellulose, hydroxyalkyl cellulose, hydroxyalkyl alkylcellulose, and polyethylene glycol of average molecular weight less than about 360 Dalton. In some embodiments, the pharmaceutical composition contains a therapeutic peptide or a pharmaceutically acceptable salt thereof; an alkylsaccharide, wherein the alkylsaccharide consists of an alkyl chain length from 8 to 16 carbon atoms (e.g., 12 carbon atoms) linked by glycosidic linkage to a maltose; a pharmaceutically acceptable excipient selected from the group consisting of alkylcellulose, hydroxyalkyl cellulose, hydroxyalkyl alkylcellulose, and polyethylene glycol of average molecular weight less than about 360 Dalton; and a tonicity agent. In some embodiments, the pharmaceutical composition contains a therapeutic peptide or a pharmaceutically acceptable salt thereof; an alkylsaccharide, wherein the alkylsaccharide consists of an alkyl chain length from 8 to 16 carbon atoms (e.g., 12 carbon atoms) linked by glycosidic linkage to a maltose; a pharmaceutically acceptable excipient selected from the group consisting of alkylcellulose, hydroxyalkyl cellulose, hydroxyalkyl alkylcellulose, and polyethylene glycol of average molecular weight less than about 360 Dalton; and a buffering agent. In some embodiments, the pharmaceutical composition contains a therapeutic peptide or a pharmaceutically acceptable salt thereof; an alkylsaccharide, wherein the alkylsaccharide consists of an alkyl chain length from 8 to 16 carbon atoms (e.g., 12 carbon atoms) carbon atoms linked by glycosidic linkage to a maltose; a pharmaceutically acceptable excipient selected from the group consisting of alkylcellulose, hydroxyalkyl cellulose, hydroxyalkyl alkylcellulose, and polyethylene glycol of average molecular weight less than about 360 Dalton; a tonicity agent; and a buffering agent.

In some embodiments, the pharmaceutical composition contains a therapeutic peptide or a pharmaceutically acceptable salt thereof; DDM; and METHOCEL™ 4000. In some embodiments, the pharmaceutical composition contains a therapeutic peptide or a pharmaceutically acceptable salt thereof; DDM; and METHOCEL™ E4M. In some embodiments, the pharmaceutical composition contains a therapeutic peptide or a pharmaceutically acceptable salt thereof; DDM; and PEG 200. In some embodiments, the pharmaceutical composition contains a therapeutic peptide or a pharmaceutically acceptable salt thereof; DDM; METHOCEL™ 4000; and mannitol. In some embodiments, the pharmaceutical composition contains a therapeutic peptide or a pharmaceutically acceptable salt thereof; DDM; METHOCEL™ E4M; and mannitol. In some embodiments, the pharmaceutical composition contains a therapeutic peptide or a pharmaceutically acceptable salt thereof; DDM; PEG 200; and mannitol. In some embodiments, the pharmaceutical composition contains a therapeutic peptide or a pharmaceutically acceptable salt thereof; DDM; METHOCEL™ 4000; mannitol; and acetate buffer. In some embodiments, the pharmaceutical composition contains a therapeutic peptide or a pharmaceutically acceptable salt thereof; DDM; METHOCEL™ E4M; mannitol; and acetate buffer. In some embodiments, the pharmaceutical composition contains a therapeutic peptide or a pharmaceutically acceptable salt thereof; DDM; PEG 200; mannitol; and acetate buffer.

In some embodiments, the pharmaceutical composition contains octreotide or a pharmaceutically acceptable salt thereof; DDM; and METHOCEL™ 4000. In some embodiments, the pharmaceutical composition contains octreotide or a pharmaceutically acceptable salt thereof; DDM; and METHOCEL™ E4M. In some embodiments, the pharmaceutical composition contains octreotide or a pharmaceutically acceptable salt thereof; DDM; and PEG 200. In some embodiments, the pharmaceutical composition contains octreotide or a pharmaceutically acceptable salt thereof; DDM; METHOCEL™ 4000; and mannitol. In some embodiments, the pharmaceutical composition contains octreotide or a pharmaceutically acceptable salt thereof; DDM; METHOCEL™ E4M; and mannitol. In some embodiments, the pharmaceutical composition contains octreotide or a pharmaceutically acceptable salt thereof; DDM; PEG 200; and mannitol. In some embodiments, the pharmaceutical composition contains octreotide or a pharmaceutically acceptable salt thereof; DDM; METHOCEL™ 4000; mannitol; and acetate buffer. In some embodiments, the pharmaceutical composition contains octreotide or a pharmaceutically acceptable salt thereof; DDM; METHOCEL™ E4M; mannitol; and acetate buffer. In some embodiments, the pharmaceutical composition contains octreotide or a pharmaceutically acceptable salt thereof; DDM; PEG 200; mannitol; and acetate buffer.

In some embodiments, the pharmaceutical composition contains lanreotide or a pharmaceutically acceptable salt thereof; DDM; and METHOCEL™ 4000. In some embodiments, the pharmaceutical composition contains lanreotide or a pharmaceutically acceptable salt thereof; DDM; and METHOCEL™ E4M. In some embodiments, the pharmaceutical composition contains lanreotide or a pharmaceutically acceptable salt thereof; DDM; and PEG 200. In some embodiments, the pharmaceutical composition contains lanreotide or a pharmaceutically acceptable salt thereof; DDM; METHOCEL™ 4000; and mannitol. In some embodiments, the pharmaceutical composition contains lanreotide or a pharmaceutically acceptable salt thereof; DDM; METHOCEL™ E4M; and mannitol. In some embodiments, the pharmaceutical composition contains lanreotide or a pharmaceutically acceptable salt thereof; DDM; PEG 200; and mannitol. In some embodiments, the pharmaceutical composition contains lanreotide or a pharmaceutically acceptable salt thereof; DDM; METHOCEL™ 4000; mannitol; and acetate buffer. In some embodiments, the pharmaceutical composition contains lanreotide or a pharmaceutically acceptable salt thereof; DDM; METHOCEL™ E4M; mannitol; and acetate buffer. In some embodiments, the pharmaceutical composition contains lanreotide or a pharmaceutically acceptable salt thereof; DDM; PEG 200; mannitol; and acetate buffer.

In some embodiments, the pharmaceutical composition contains pasireotide or a pharmaceutically acceptable salt thereof; DDM; and METHOCEL™ 4000. In some embodiments, the pharmaceutical composition contains pasireotide or a pharmaceutically acceptable salt thereof; DDM; and METHOCEL™ E4M. In some embodiments, the pharmaceutical composition contains pasireotide or a pharmaceutically acceptable salt thereof; DDM; and PEG 200. In some embodiments, the pharmaceutical composition contains pasireotide or a pharmaceutically acceptable salt thereof; DDM; METHOCEL™ 4000; and mannitol. In some embodiments, the pharmaceutical composition contains pasireotide or a pharmaceutically acceptable salt thereof; DDM; METHOCEL™ E4M; and mannitol. In some embodiments, the pharmaceutical composition contains pasireotide or a pharmaceutically acceptable salt thereof; DDM; PEG 200; and mannitol. In some embodiments, the pharmaceutical composition contains pasireotide or a pharmaceutically acceptable salt thereof; DDM; METHOCEL™ 4000; mannitol; and acetate buffer. In some embodiments, the pharmaceutical composition contains pasireotide or a pharmaceutically acceptable salt thereof; DDM; METHOCEL™ E4M; mannitol; and acetate buffer. In some embodiments, the pharmaceutical composition contains pasireotide or a pharmaceutically acceptable salt thereof; DDM; PEG 200; mannitol; and acetate buffer.

Pharmaceutical compositions provided herein may be highly stable and have long shelf-life. In some embodiments, the pharmaceutical composition is stable for at least 1 month, at least 2 months, at least 3 months, at least 4 months, at least 5 months, or at least 6 months when stored at temperatures from about 25 to about 40 degrees Celcius. In some embodiments, the pharmaceutical composition is stable for at least 6 months, at least 9 months, at least 12 months, at least 18 months, at least 24 months, at least 30 months, or at least 36 months when stored at temperatures from about 2 to about 8 degrees Celsius.

Stability can be determined by measuring the appearance of degradation products in the pharmaceutical composition by methods known in the art. One such method is to measure the areas of degradation peaks in an HPLC trace (e.g., as detected by UV absorbance) at particular time points after storage relative to the area of the octreotide peak. In some embodiments, the total percentage of degradation products in the pharmaceutical composition after 2 weeks of storage at 40 degrees Celsius is less than about 3%, less than about 2.5%, less than about 2%, less than about 1.5%, less than about 1%, less than about 0.5%, less than about 0.25%, or less than about 0.1%. In some embodiments, the total percentage of degradation products in the pharmaceutical composition after 4 weeks of storage at 40 degrees Celsius is less than about 6%, less than about 5.5%, less than about 5%, less than about 4.5%, less than about 4%, less than about 3.5%, less than about 3%, less than about 2.5%, less than about 2%, less than about 1.5%, less than about 1%, less than about 0.5%, less than about 0.25%, or less than about 0.1%. In some embodiments, the total percentage of degradation products in the pharmaceutical composition after 2 weeks of storage at 25 degrees Celsius is less than about 3%, less than about 2.5%, less than about 2%, less than about 1.5%, less than about 1%, less than about 0.5%, less than about 0.25%, or less than about 0.1%. In some embodiments, the total percentage of degradation products in the pharmaceutical composition after 4 weeks of storage at 25 degrees Celsius is less than about 6%, less than about 5.5%, less than about 5%, less than about 4.5%, less than about 4%, less than about 3.5%, less than about 3%, less than about 2.5%, less than about 2%, less than about 1.5%, less than about 1%, less than about 0.5%, less than about 0.25%, or less than about 0.1%. In some embodiments, the total percentage of degradation products in the pharmaceutical composition after 2 weeks of storage at 5 degrees Celsius is less than about 3%, less than about 2.5%, less than about 2%, less than about 1.5%, less than about 1%, less than about 0.5%, less than about 0.25%, or less than about 0.1%. In some embodiments, the total percentage of degradation products in the pharmaceutical composition after 4 weeks of storage at 25 degrees Celsius is less than about 6%, less than about 5.5%, less than about 5%, less than about 4.5%, less than about 4%, less than about 3.5%, less than about 3%, less than about 2.5%, less than about 2%, less than about 1.5%, less than about 1%, less than about 0.5%, less than about 0.25%, or less than about 0.1%.

Pharmaceutical compositions provided herein may have high levels of bioavailability. Bioavailability can be measured by methods known in the art and can be described by, for example, peak plasma concentration (Cₘₐₓ), AUC_{(0-T)}, Tₘₐₓ, or T_{1/2}. In some embodiments, the pharmaceutical composition has a Cₘₐₓ (e.g., average Cₘₐₓ) of at least about 5, at least about 10, at least about 15, at least about 20, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 ng/mL. In some embodiments, the pharmaceutical composition has an AUC_{(0-T)} (e.g., average AUC_{(0-T)}) of at least about 500, at least about 750, at least about 1000, at least about 1250, at least about 1500, at least about 2000, at least about 2500, at least about 3000, at least about 3500, at least about 4000, at least about 4500, or at least about 5000 ng·min/mL.

For certain therapeutic peptides, such as octreotide, subcutaneous administration results in nearly 100% of the peptide being present in the blood stream. Bioavailability of pharmaceutical compositions of the same therapeutic peptide for use via other routes of administration can be measured as a percentage of the bioavailability of the subcutaneously administered formulation. For example, bioavailability can be determined by measuring the relative AUC_{(0-T)} (e.g., average AUC_{(0-T)} or Cₘₐₓ (e.g., average Cₘₐₓ) or an intranasally administered composition compared to the AUC_{(0-T)} (e.g., average AUC_{(0-T)} or Cₘₐₓ (e.g., average Cₘₐₓ) for a corresponding subcutaneously administered composition. In some embodiments, the AUC_{(0-T)} (e.g., average AUC_{(0-T)}) of the pharmaceutical composition is at least 10%, at least 12%, at least 15%, at least 18%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% of the AUC_{(0-T)} (e.g., average AUC_{(0-T)} for the same dosage of the therapeutic peptide when administered subcutaneously. In some embodiments, the Cₘₐₓ (e.g., average Cₘₐₓ) of the pharmaceutical composition is at least 10%, at least 12%, at least 15%, at least 18%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% of the Cₘₐₓ (e.g., average Cₘₐₓ) for the same dosage of the therapeutic peptide when administered subcutaneously.

Additionally, the pharmaceutical compositions provided herein may have good tolerability. This may be achieved, for example, by having a physiological pH and/or by having isotonicity. In some embodiments, all of the components of the pharmaceutical composition are tolerable for intranasal administration and do not cause pain, excessive tearing, or other undesirable effects upon administration.

In some embodiments, the pharmaceutical composition provided herein has properties of stability, bioavailability, and tolerability as described herein.

### Methods of Preparation

The pharmaceutical compositions described herein may be produced and evaluated generally as described below and known to those of skill in the art. Additionally, the skilled artisan, based on the teachings provided herein and the particular composition to be prepared will also be able to modify the preparation methods described herein and known in the art without undue experimentation.

Generally, the pharmaceutical compositions provided herein can be prepared by dissolving (e.g., sequentially (in any appropriate order) or simultaneously) sufficient quantities of a therapeutic peptide (or a pharmaceutically acceptable salt thereof), alkylsaccharide, excipient, and any other components in a sufficient volume of water (e.g., deionized water) to achieve the desired concentration of therapeutic peptide and other components. In some embodiments, therapeutic peptide (or a pharmaceutically acceptable salt thereof), alkylsaccharide, and excipient are sequentially dissolved in water. In some embodiments, therapeutic peptide (or a pharmaceutically acceptable salt thereof), excipient, and alkylsaccharide are sequentially dissolved in water. In some embodiments, alkylsaccharide, excipient, and therapeutic peptide (or a pharmaceutically acceptable salt thereof) are sequentially dissolved in water. In some embodiments, alkylsaccharide, therapeutic peptide (or a pharmaceutically acceptable salt thereof), and excipient are sequentially dissolved in water. In some embodiments, excipient, alkylsaccharide, and therapeutic peptide (or a pharmaceutically acceptable salt thereof) are sequentially dissolved in water. In some embodiments, excipient, therapeutic peptide (or a pharmaceutically acceptable salt thereof), and alkylsaccharide are sequentially dissolved in water. Other components of the formulation may be added at appropriate stages of the process.

Dissolution may be aided by stirring, swirling, heating, etc., including combinations of two or more of the foregoing. Methods known in the art may be used to adjust the pH of the solution, if needed. The above protocol may be undertaken in sterile conditions and in accordance with GMP and GLP (Good Laboratory Practice) standards and, when intended for administration to humans, should also conform to regulatory guidelines.

Analysis may be performed by techniques including, without limitation, HPLC, spectrophotometry, LC/MS/MS, and mass spectrometry.

### Methods of Use

The pharmaceutical compositions described herein may be used to treat or prevent a condition in an individual. Conditions that may be treated or prevented using a pharmaceutical composition provided herein include, without limitation, acromegaly, carcinoid tumors, vasoactive intestinal peptide secreting tumors, diarrhea associated with acquired immune deficiency syndrome (AIDS), diarrhea associated with chemotherapy, diarrhea associated with radiation therapy, dumping syndrome, adrenal gland neuroendocrine tumors, bowel obstruction, enterocutaneous fistulae, gastrinoma, acute bleeding of gastroesophageal varices, islet cell tumors, lung neuroendocrine tumors, malignancy, meningiomas, gastrointestinal tract neuroendocrine tumors, thymus neuroendocrine tumors, pancreatic fistulas, pancreas neuroendocrine tumors, pituitary adenomas, short-bowel syndrome, small or large cell neuroendocrine tumors, thymomas and thymic carcinomas, Zollinger Ellison syndrome, acute pancreatitis, breast cancer, chylothorax, congenital lymphedema, diabetes mellitus, gastric paresis, hepatocellular carcinoma, non-variceal upper gastrointestinal bleeding, obestity, pancreaticoduodenectomy, prostate cancer, protein-losing enteropathy, small cell lung cancer, thyroid cancer, thyroid eye disease, vascular (arterio-venous) malformations of the gastrointestinal tract, polycystic kidney disease, Cushing's disease, GHRH-producing tumors, and other conditions resulting in abnormally elevated growth hormone, insulin, or glucagon levels.

In some embodiments, the pharmaceutical composition is used to treat acromegaly, carcinoid tumors, or vasoactive intestinal peptide secreting tumors.

In some embodiments, provided is a method for treating or preventing a condition described herein, comprising administering to an individual in need thereof a therapeutically effective amount of a pharmaceutical composition provided herein.

In some embodiments, provided is a pharmaceutical composition provided herein for use in treating or preventing a condition described herein in an individual.

In some embodiments, provided is the use of a pharmaceutical composition provided herein in the treatment or prevention of a condition described herein in an individual.

In some embodiments, provided is the use of a pharmaceutical composition provided herein in the manufacture of a medicament for the treatment or prevention of a condition described herein in an individual.

### Administration and Dosing Regimes

The pharmaceutical compositions described herein may be administered orally, sublingually, subcutaneously, intravenously, intranasally, topically, transdermally, intraperitoneally, intramuscularly, intrapulmonarilly, vaginally, rectally, or intraocularly to an individual. In a particular embodiment, the pharmaceutical composition is administered intranasally.

The pharmaceutical compositions described herein may be used alone or in conjunction with (e.g., prior to, concurrently with, or after) other modes of treatments (e.g., adjunctive therapy with additional pharmaceutical agents) used to treat or prevent the condition being treated/prevented and/or administration of an additional treatment modality, or combinations of the foregoing. As used herein, the term “additional treatment modality” refers to treatment/prevention of the conditions described herein without the use of a pharmaceutical agent (e.g., psychotherapy, occupational therapy, surgery, etc.). Where combinations of pharmaceutical agent(s) and/or additional treatment modality(ies) are used, they may be, independently, administered prior to, concurrently with, or after administration of a pharmaceutical composition described herein.

The optimal combination of one or more additional treatment modalities and/or additional pharmaceutical agents in conjunction with administration of the compositions described herein, can be determined by an attending physician or veterinarian based on the individual and taking into consideration the various factors effecting the particular individual, including those described herein.

The compositions described herein will generally be used in an amount effective to achieve the intended result, for example in an amount effective to treat or prevent the particular condition being treated or prevented. The compositions may be administered therapeutically to achieve therapeutic benefit. The term “therapeutic benefit” as used herein refers to eradication or amelioration of the underlying condition being treated and/or eradication or amelioration of one or more of the symptoms associated with the underlying condition such that the individual reports an improvement in feeling or condition, notwithstanding that the individual may still be afflicted with the underlying condition. Therapeutic benefit may include halting or slowing the progression of the condition, regardless of whether improvement is realized.

The amount of the composition administered in order to administer an effective amount will depend upon a variety of factors, including, for example, the particular condition being treated, the frequency of administration, the particular pharmaceutical composition being administered, the severity of the condition being treated and the age, weight and general health of the individual, the adverse effects experienced by the individual being treated, etc.

The amount of active ingredient in the composition to produce a single dosage form will vary depending upon the individual to which the active ingredient is administered and the particular mode of administration. It will be understood, however, that the specific dose level for any particular individual will depend upon a variety of factors including the activity of the specific compound employed, the age, body weight, body area, body mass index (BMI), general health, sex, diet, time of administration, route of administration, rate of excretion, drug combination, and the type, progression, and severity of the particular disease undergoing therapy. The pharmaceutical unit dosage chosen is usually fabricated and administered to provide a defined final concentration of drug in the blood, tissues, organs, or other targeted region of the body.

The pharmaceutical composition may be administered monthly, biweekly, weekly, twice a week, three times a week, four times a week, five time a week, six times a week, once a day, twice a day, three times a day, four times a day, five times a day, six times a day, seven times a day, eight times a day, nine times a day, or ten times a day. The dosage of the therapeutic peptide may be about 10 mcg/day, about 50 mcg/day, about 100 mcg/day, about 150 mcg/day, about 200 mcg/day, about 250 mcg/day, about 300 mcg/day, about 350 mcg/day, about 400 mcg/day, about 450 mcg/day, about 500 mcg/day, about 550 mcg/day, about 600 mcg/day, about 650 mcg/day, about 700 mcg/day, about 750 mcg/day, about 800 mcg/day, about 850 mcg/day, about 900 mcg/day, about 950 mcg/day, about 1000 mcg/day, about 1100 mcg/day, about 1200 mcg/day, about 1300 mcg/day, about 1400 mcg/day, about 1500 mcg/day, about 1600 mcg/day, about 1700 mcg/day, about 1800 mcg/day, about 1900 mcg/day, about 2 mg/day, about 3 mg/day, about 4 mg/day, about 6 mg/day, about 8 mg/day, about 10 mg/day, about 12 mg/day, about 14 mg/day, about 16 mg/day, about 18 mg/day, about 20 mg/day, about 22 mg/day, about 24 mg/day, about 26 mg/day, about 28 mg/day, about 30 mg/day, about 40 mg/day, or about 50 mg/day.

The compositions provided herein and the other therapeutically active agents can be administered at the recommended maximum clinical dosage or at lower doses. Dosage levels of the therapeutic peptide in the compositions provided herein may be varied so as to obtain a desired therapeutic response depending on the route of administration, severity of the disease and the response of the individual. When administered in combination with other pharmaceutical agents, the pharmaceutical agents can be formulated as separate compositions that are given at the same time or different times, or the pharmaceutical agents can be given as a single composition.

In some embodiments, the viscosity of the pharmaceutical composition may be about 1 cP, about 2 cPs, about 3 cPs, about 4 cPs, about 5 cPs, about 6 cPs, about 7 cPs, 8 cPs, about 9 cPs, about 10 cPs, about 11 cPs, about 12 cPs, about 13 cPs, about 14 cPs, about 15 cPs, about 16 cPs, about 17 cPs, 18 cPs, about 19 cPs, about 20 cPs, about 21 cPs, about 22 cPs, about 23 cPs, about 24 cPs, or about 25 cPs. In some embodiments, the viscosity of the pharmaceutical composition may be greater than about 1 cP, about 2 cPs, about 3 cPs, about 4 cPs, about 5 cPs, about 6 cPs, about 7 cPs, 8 cPs, about 9 cPs, about 10 cPs, about 11 cPs, about 12 cPs, about 13 cPs, about 14 cPs, about 15 cPs, about 16 cPs, about 17 cPs, 18 cPs, about 19 cPs, about 20 cPs, about 21 cPs, about 22 cPs, about 23 cPs, or about 24 cPs. In some embodiments, the viscosity of the pharmaceutical composition may be less than about 2 cPs, about 3 cPs, about 4 cPs, about 5 cPs, about 6 cPs, about 7 cPs, 8 cPs, about 9 cPs, about 10 cPs, about 11 cPs, about 12 cPs, about 13 cPs, about 14 cPs, about 15 cPs, about 16 cPs, about 17 cPs, 18 cPs, about 19 cPs, about 20 cPs, about 21 cPs, about 22 cPs, about 23 cPs, about 24 cPs, or about 25 cPs. In some embodiments, the viscosity of the pharmaceutical composition may be between about 1 cP and about 25 cPs, between about 5 and about 20 cPs, about 5 and about 10 cPs, about 2 and about 8 cPs, about 10 and about 20 cPs or about 1 and about 5 cPs.

For certain routes of administration, such as intranasal administration, the average droplet size of the pharmaceutical composition may be in a particular size rage as appropriate for that route of administration. For instance, the droplet size may be sufficient to substantially prevent the composition from being delivered into the lungs. In some embodiments, the average droplet size of the pharmaceutical composition is about 10 μm, about 20 μm, about 30 μm, about 40 μm, about 50 μm, about 60 μm, about 70 μm, about 80 μm, about 90 μm, about 100 μm, about 150 μm, about 200 μm, about 250 μm, about 300 μm, about 350 μm, about 400 μm, about 450 μm, about 500 μm, about 550 μm, about 600 μm, about 650 μm, about 700 μm, about 750 μm, about 800 μm, about 850 μm, about 900 μm, about 950 μm, or about 1000 μm. In some embodiments, the average droplet size of the pharmaceutical composition is greater than about 10 μm, about 20 μm, about 30 μm, about 40 μm, about 50 μm, about 60 μm, about 70 μm, about 80 μm, about 90 μm, about 100 μm, about 150 μm, about 200 μm, about 250 μm, about 300 μm, about 350 μm, about 400 μm, about 450 μm, about 500 μm, about 550 μm, about 600 μm, about 650 μm, about 700 μm, about 750 μm, about 800 μm, about 850 μm, about 900 μm, or about 950 μm. In some embodiments, the average droplet size of the pharmaceutical composition is less than about 15 μm, about 20 μm, about 30 μm, about 40 μm, about 50 μm, about 60 μm, about 70 μm, about 80 μm, about 90 μm, about 100 μm, about 150 μm, about 200 μm, about 250 μm, about 300 μm, about 350 μm, about 400 μm, about 450 μm, about 500 μm, about 550 μm, about 600 μm, about 650 μm, about 700 μm, about 750 μm, about 800 μm, about 850 μm, about 900 μm, about 950 μm, or about 1000 μm. In some embodiments, the average droplet size of the pharmaceutical composition is between about 10 μm, and about 20 μm, between about 10 μm and about 50 μm, between about 20 μm and about 100 μm, between about 10 μm and about 1000 μm, or between about 100 μm and about 500 μm.

### Kits

Also provided are articles of manufacture and kits containing the pharmaceutical compositions provided herein. The article of manufacture may comprise a container with a label. Suitable containers include, for example, bottles, vials, and test tubes. The containers may be formed from a variety of materials such as glass or plastic. The container may hold a pharmaceutical composition provided herein. The label on the container may indicate that the pharmaceutical composition is used for treating or suppressing a condition described herein, and may also indicate directions for either in vivo or in vitro use.

Also provided are kits comprising any one or more of the compositions described herein. In some embodiments, the kit comprises the container described above. In other embodiments, the kit comprises the container described above and a second container comprising a buffer. It may further include other materials desirable from a commercial and user standpoint, including other buffers, diluents, filters, needles, syringes, and package inserts with instructions for performing any methods described herein.

In certain embodiments the kits may include a dosage amount of at least one pharmaceutical composition as disclosed herein. Kits may also comprise a means for the delivery of the pharmaceutical composition.

The kits may include other pharmaceutical agents for use in conjunction with the pharmaceutical composition described herein. These pharmaceutical agents may be provided in a separate form, or mixed with the pharmaceutical composition described herein, provided such mixing does not reduce the effectiveness of either the pharmaceutical agent or pharmaceutical composition described herein and is compatible with the route of administration. Similarly, the kits may include additional agents for adjunctive therapy or other agents effective in the treatment or prevention of the conditions described herein.

The kits may optionally include appropriate instructions for preparation and administration of the pharmaceutical composition, side effects of the pharmaceutical composition, and any other relevant information. The instructions may be in any suitable format, including, but not limited to, printed matter, videotape, computer readable disk, optical disc or directions to internet-based instructions.

Kits may also be provided that contain sufficient dosages of the pharmaceutical compositions described herein to provide effective treatment for an individual for an extended period, such as 1-3 days, 1-5 days, a week, 2 weeks, 3, weeks, 4 weeks, 6 weeks, 8 weeks, 3 months, 4 months, 5 months, 6 months, 7 months, 8 months, 9 months or more.

Kits may also include multiple doses of the pharmaceutical composition and instructions for use and may be packaged in quantities sufficient for storage and use in pharmacies, for example, hospital pharmacies and compounding pharmacies.

The kits may include the pharmaceutical composition as described herein packaged in either a unit dosage form or in a multi-use form. The kits may also include multiple units of the unit dose form.

In certain embodiments are provided a pharmaceutical composition described herein in a unit dose form. In other embodiments a pharmaceutical composition may be provided in a multi-dose form.

### Examples

The following examples are offered to illustrate but not to limit the compositions, uses, and methods provided herein.

### Example 1: Preparation of Octreotide Compositions

Octreotide compositions were prepared by the following procedure. A vessel was placed on a mixer, and to the vessel was added 95% of the target amount of water. The target amount of buffering agent was added to the vessel. Then the target amount of excipient was slowly added to the vessel. Mixing was continued until the excipient was visually dissolved. The target amount of octreotide acetate was added to the vessel, and mixing was continued until the octreotide acetate was visually dissolved. The target amount of tonicity agent was added to the vessel, and mixing was carried out for 10 minutes. The target amount of DDM that had equilibrated at room temperature for at least 2 hours was added to the vessel, and mixing was continued for at least 25 minutes. The pH of the solution in the vessel was measured and adjusted if necessary. Additional water was added until the target amount of water was reached.

### Example 2: Octreotide Bioavailability Following Intranasal and Subcutaneous Administration

To determine the plasma pharmacokinetics of octreotide following intranasal and subcutaneous administration of octreotide formulations, five octreotide formulations as provided in Table 1 were prepared as described in Example 1.

TABLE 1

| Formulation | Ingredients | Physical characterization |
|---|---|---|
| Formulation #1 | Octreotide (4 mg/mL) in | White liquid |
| | acetate, mannitol, Avicel | |
| | RC-591, DDM, pH = 5.5 | |
| Formulation #2 | Octreotide (4 mg/mL) in | Clear, colorless liquid |
| | acetate, mannitol, Methocel | essentially free from |
| | 4000, DDM, pH = 5.5 | visible particles |
| Formulation #3 | Octreotide (4 mg/mL) in | Clear, colorless liquid |
| | acetate, mannitol, PEG400, | essentially free from |
| | DDM, pH = 5.5 | visible particles |
| Formulation #4 | Octreotide (4 mg/mL) in | Clear, colorless liquid |
| | acetate, mannitol, DDM, | essentially free from |
| | pH = 5.5 | visible particles |
| Formulation #5 | Octreotide (0.25 mg/mL) in | Clear, colorless liquid |
| | acetate, mannitol, DDM, | essentially free from |
| | pH = 5.5 | visible particles |

Five groups of male and female New Zealand White rabbits were administered the test formulations as summarized in Table 2.

TABLE 2

| | | | | Dose | | |
|---|---|---|---|---|---|---|
| | | | | Concentration | | Number of |
| | Route of | | Dose Vol | (octreotide | | Animals and |
| Group | Administration | Formulation | (μl) | acetate) | | Sex |
| 1 | Intranasal | 1 | 100 μl (50 μl | 4 | mg/mL | 3M/3F |
| | | | per nostril) | | | |
| 2 | Intranasal | 2 | 100 μl (50 μl | 4 | mg/mL | 3M/3F |
| | | | per nostril) | | | |
| 3 | Intranasal | 3 | 100 μl (50 μl | 4 | mg/mL | 3M/3F |
| | | | per nostril) | | | |
| 4 | Intranasal | 4 | 100 μl (50 μl | 4 | mg/mL | 3M/3F |
| | | | per nostril) | | | |
| 5 | Subcutaneous | 5 | 500 μl | 0.25 | mg/mL | 3M/3F |

During acclimation, animals were conditioned to intranasal dosing by single installations of 50 μL saline into both nares three times a day for three consecutive days. Groups 1-4 were dosed intranasally via instillation of 50 μL into both nares using a Pipetteman and disposable plastic tip. Rabbits in Group 5 were dosed subcutaneously via bolus injection into the nape of the neck using a sterile stainless steel needle attached to a sterile glass syringe. Following dose administration, ten serial blood samples (˜2.0 ml each) were collected by direct venipuncture from a marginal ear vein and deposited into prelabeled 2-ml draw tubes containing K₂EDTA as the anticoagulant at the following times relative to dosing: ˜−30 (predose), 5, 10, 20, 30, 40, 60, 90, 120, and 150 minutes. The total volume collected did not exceed 1% of body weight for the animals.

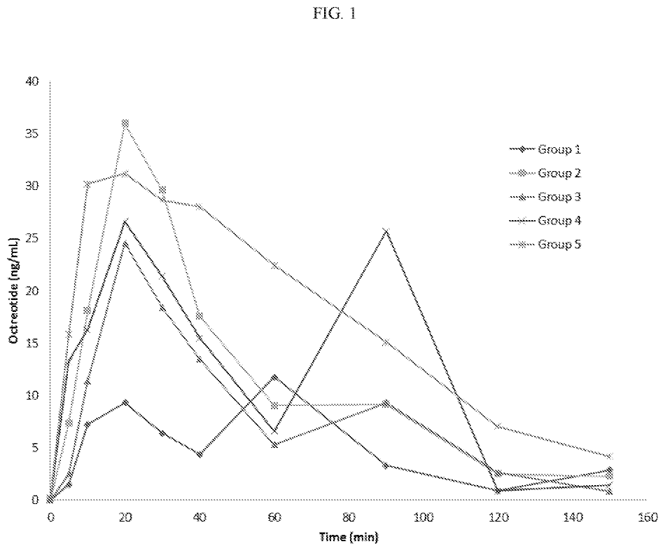

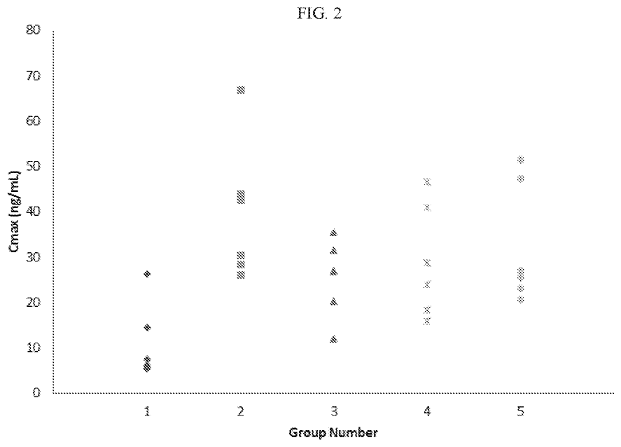

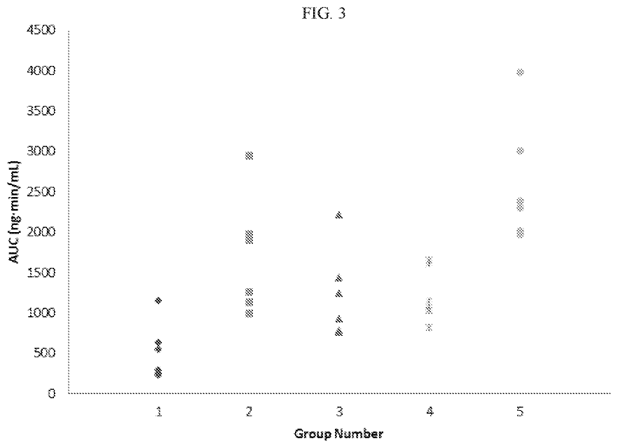

Data analysis consisted of recording and documenting animal body weights, dosing details, animal observations, and blood sampling times (actual and elapsed). Bioanalysis of rabbit plasma samples for octreotide and pharmacokinetic analysis of the octreotide plasma concentration-time data are provided in Tables 3-5 and FIGS. 1-3. Cₘₐₓ is the observed maximum plasma concentration after dosing. Tₘₐₓ is the time Cₘₐₓ is reached. T_{1/2} is apparent plasma terminal half-life calculated by ln(2)/λ, where λ represents the elimination rate constant for the log-linear portion of the terminal phase. AUC_{(0-T)} is the area under the plasma concentration-time curve from time 0 to the last measurable plasma concentration.

TABLE 3

| | Cₘₐₓ | Tₘₐₓ | AUC(_{0-T}) | T_{1/2} |
|---|---|---|---|---|
| Formulation | (ng/mL) | (min) | (ng · min/mL) | (min) |
| #1 Female | 13.3 ± 11.2 | 43.3 ± 40.4 | 634 ± 472 | 20.3 ± 4.11 |
| #1 Male | 7.29 ± 6.54 | 43.3 ± 40.4 | 332 ± 274 | 17.5 ± 6.01 |
| #2 Female | 28.2 ± 2.20 | 16.7 ± 5.77 | 1,130 ± 132 | 25.1 ± 11.2 |
| #2 Male | 48.6 ± 16.4 | 23.3 ± 5.77 | 1,930 ± 40.0 | 35.8 ± 12.2 |
| #3 Female | 27.6 ± 7.62 | 20.0 ± 0.00 | 980 ± 250 | 35.2 ± 10.1 |
| #3 Male | 22.5 ± 9.23 | 26.7 ± 11.6 | 1,140 ± 513 | 33.7 ± 9.61 |
| #4 Female | 38.8 ± 9.16 | 11.7 ± 7.64 | 1,380 ± 315 | 18.8 ± 3.17 |
| #4 Male | 19.5 ± 4.11 | 23.3 ± 5.77 | 989 ± 155 | 24.4 ± 6.87 |
| #5 Female | 23.6 ± 3.19 | 23.3 ± 15.3 | 2,100 ± 177 | 47.0 ± 8.62 |
| #5 Male | 41.5 ± 14.0 | 23.3 ± 15.3 | 3,130 ± 804 | 25.4 ± 3.61 |

TABLE 4

| Group # | | #1(ng/mL) | #2(ng/mL) | #3(ng/mL) | #4(ng/mL) | #5(ng/mL) |
|---|---|---|---|---|---|---|
| 0 | | 0 | 0 | 0 | 0.16 | 0.33 |
| 5 | min | 1.48 | 7.37 | 2.5 | 13.28 | 15.80 |
| 10 | min | 7.21 | 18.06 | 11.4 | 16.30 | 30.22 |
| 20 | min | 9.32 | 36.02 | 24.6 | 26.62 | 31.18 |
| 30 | min | 6.39 | 29.65 | 18.4 | 21.42 | 28.65 |
| 40 | min | 4.35 | 17.54 | 13.5 | 15.47 | 28.02 |
| 60 | min | 11.76 | 9.03 | 5.3 | 6.57 | 22.43 |
| 90 | min | 3.30 | 9.14 | 9.3 | 25.69 | 15.10 |
| 120 | min | 0.87 | 2.49 | 2.6 | 0.86 | 7.02 |
| 150 | min | 2.89 | 2.27 | 0.8 | 1.44 | 4.16 |

TABLE 5

| Group # | AUC | CV% | % of SC | Cₘₐₓ | CV% | % of SC |
|---|---|---|---|---|---|---|
| #1 Average | 746.18 | 89.34 | 8.9 | 19.92 | 106.75 | 19.1 |
| #2 Average | 1700.81 | 42.94 | 20.3 | 39.67 | 38.47 | 38.1 |
| #3 Average | 1231.19 | 44.85 | 14.7 | 25.50 | 32.90 | 24.5 |
| #4 Average | 1883.78 | 63.72 | 22.5 | 42.82 | 61.50 | 41.1 |
| #5 Average | 8357.95 | 29.40 | 100.0 | 104.05 | 41.07 | 100.0 |

### Example 3: Octreotide Bioavailability Following Intranasal Administration

To determine the plasma pharmacokinetics of octreotide following intranasal administration of octreotide formulations, another six octreotide formulations as provided in Table 6 were prepared as described in Example 1.

TABLE 6

| Formulation # | Ingredients |
|---|---|
| Formulation #1 | 0.4% Octreotide Acetate, |
| | 0.25% Monocaprin/0.8% |
| | Tween 80 |
| Formulation #2 | 0.4% Octreotide Acetate, |
| | 0.18% DDM, 3% mPEG 350 |
| Formulation #3 | 0.4% Octreotide Acetate, |
| | 0.18% DDM, 0.5% Methocel |
| | 4000 |
| Formulation #4 | 0.4% Octreotide Acetate, |
| | 0.18% DDM, 5% PEG 200 |
| Formulation #5 | 0.4% Octreotide Acetate, |
| | 0.18% DDM |
| Formulation #6 | 0.4% Octreotide Acetate, |
| | 0.20% DDM, 0.1% |
| | Disodium EDTA |

Six groups of male and female New Zealand White rabbits were administered the test formulations as summarized in Table 7.

TABLE 7

| | | | Number of |
|---|---|---|---|
| | Route of | Formulation | Animals and |
| Group | Administration | # | Sex |
| 1 | Intranasal | 1 | 2M/2F |
| 2 | Intranasal | 2 | 2M/2F |
| 3 | Intranasal | 3 | 2M/2F |
| 4 | Intranasal | 4 | 2M/2F |
| 5 | Intranasal | 5 | 2M/2F |
| 6 | Intranasal | 6 | 2M/2F |

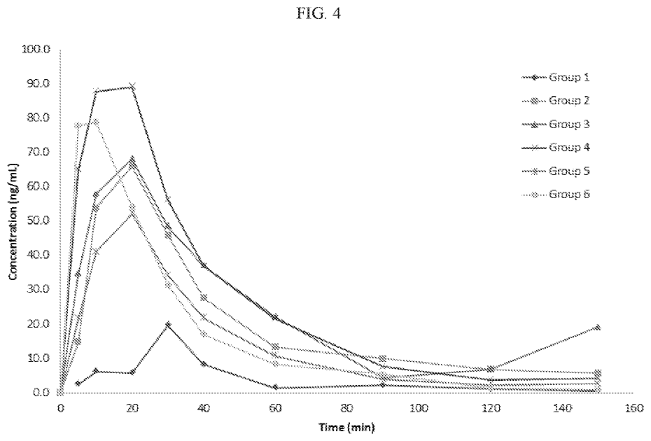

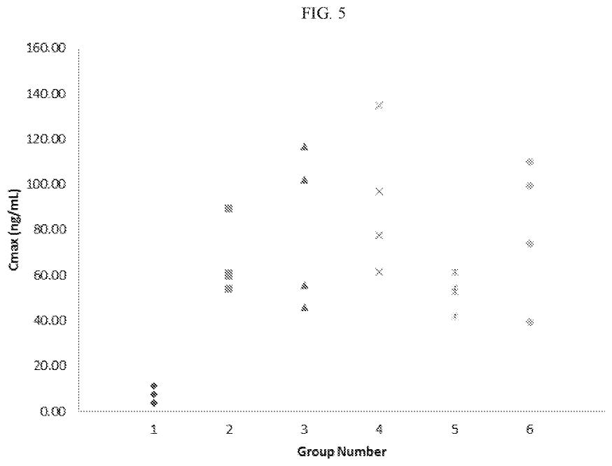

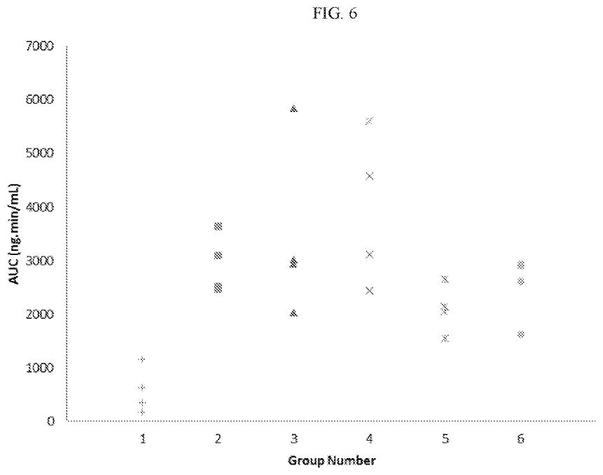

Plasma samples were collected and analyzed as described as above. Bioanalysis of rabbit plasma samples for octreotide and pharmacokinetic analysis of the octreotide plasma concentration-time data are provided in Table 8 and FIGS. 4-6. Table 8 shows the mean octreotide plasma concentration-time data for each group.

TABLE 8

| | #1 | #2 | #3 | #4 | #5 | #6 |
|---|---|---|---|---|---|---|
| Group # | (ng/mL) | (ng/mL) | (ng/mL) | (ng/mL) | (ng/mL) | (ng/mL) |
| 0 | 0 | 0 | 0 | 0 | 0 | 0 |
| 5 min | 2.4 | 14.9 | 34.6 | 65.0 | 21.6 | 77.9 |
| 10 min | 6.2 | 53.8 | 57.9 | 87.8 | 41.0 | 78.9 |
| 20 min | 5.8 | 66.1 | 68.2 | 89.1 | 52.2 | 54.2 |
| 30 min | 19.6 | 46.1 | 48.5 | 56.2 | 34.3 | 31.4 |
| 40 min | 8.2 | 27.6 | 37.0 | 36.9 | 21.8 | 17.0 |
| 60 min | 1.4 | 13.3 | 22.1 | 21.6 | 10.7 | 8.4 |
| 90 min | 2.2 | 10.0 | 4.4 | 7.6 | 4.0 | 5.4 |
| 120 min | 1.1 | 6.8 | 6.7 | 3.8 | 2.3 | 1.4 |
| 150 min | 0.5 | 5.7 | 19.0 | 4.1 | 2.5 | 1.0 |

### Example 4: Stability of Octreotide Formulations

To determine the stability of octreotide formulations, six octreotide formulations as provided in Table 9 were prepared as described in Example 1. Formulations 1-6 were all observed to be clear liquids that were essentially free of visible particles.

TABLE 9

| Formulation # | Ingredients |
|---|---|
| Formulation #1 | 0.4% Octreotide Acetate, |
| | 0.25% Monocaprin/0.8% |
| | Tween 80 |
| Formulation #2 | 0.4% Octreotide Acetate, |
| | 0.18% DDM, 3% mPEG 350 |
| Formulation #3 | 0.4% Octreotide Acetate, |
| | 0.18% DDM, 0.5% Methocel |
| | 4000 |
| Formulation #4 | 0.4% Octreotide Acetate, |
| | 0.18% DDM, 5% PEG 200 |
| Formulation #5 | 0.4% Octreotide Acetate, |
| | 0.18% DDM |
| Formulation #6 | 0.4% Octreotide Acetate, |
| | 0.20% DDM, 0.1% |
| | Disodium EDTA |

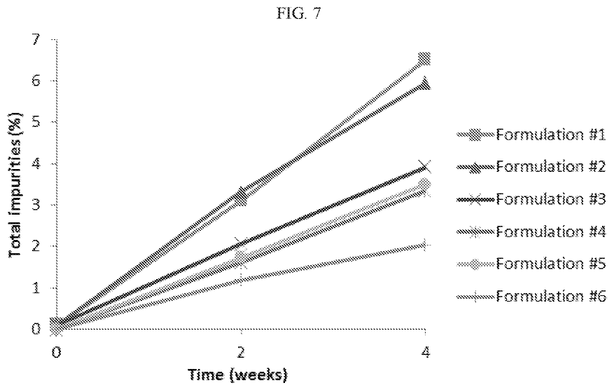

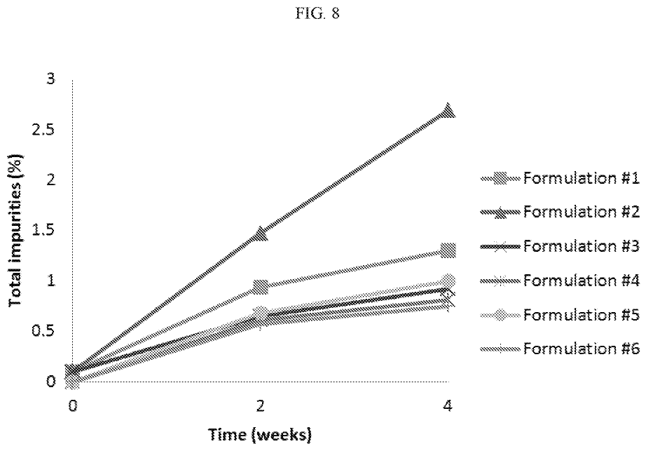

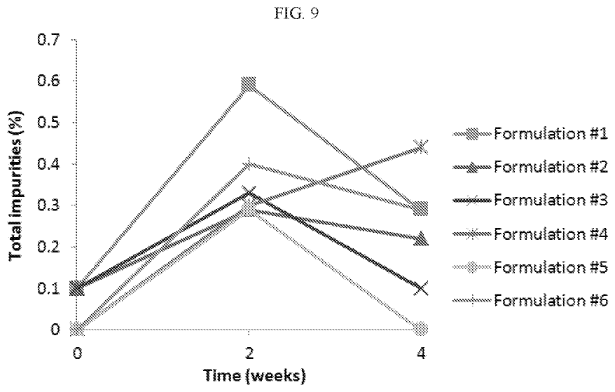

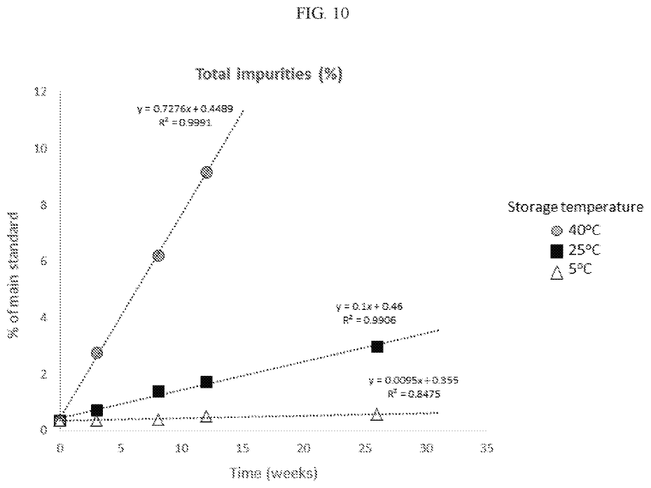

Samples of octreotide formulations in glass vials were stored at 5° C., 25° C./60% relative humidity, and 40° C./75% relative humidity for up to four weeks. Samples were measured by HPLC for degradation products after two and four weeks. The percentages of total degradation products identified by HPLC, calculated as % of total AUC for octreotide, are provided in Table 10. The data for Formulation 1-6 upon storage at 40, 25, and, 5 degrees Celsius are also shown in FIGS. 7-9, respectively. FIG. 10 shows the stability data for Formulation 3 upon storage at 40, 25, and, 5 degrees Celsius over a period of 26 weeks.

TABLE 10

| Formulation # | #1 | #2 | #3 | #4 | #5 | #6 |
|---|---|---|---|---|---|---|
| 0 weeks at 5° C. | 0.1% | 0.1% | 0.1% | 0% | 0% | 0% |
| 2 weeks at 5° C. | 0.59% | 0.29% | 0.33% | 0.3% | 0.29% | 0.4% |
| 4 weeks at 5° C. | 0.29% | 0.22% | 0.1% | 0.44% | 0% | 0.29% |
| 0 weeks at 25° C. | 0.1% | 0.1% | 0.1% | 0% | 0% | 0% |
| 2 weeks at 25° C. | 0.94% | 1.47% | 0.65% | 0.61% | 0.68% | 0.57% |
| 4 weeks at 25° C. | 1.3% | 2.69% | 0.92% | 0.81% | 1% | 0.75% |
| 0 weeks at 40° C. | 0.1% | 0.1% | 0.1% | 0% | 0% | 0% |
| 2 weeks at 40° C. | 3.11% | 3.32% | 2.06% | 1.62% | 1.73% | 1.19% |
| 4 weeks at 40° C. | 6.51% | 5.95% | 3.92% | 3.36% | 3.5% | 2.03% |

### Example 5: Stability of Octreotide Formulations

To further determine the stability of octreotide formulations, another four octreotide formulations as provided in Table 11 were prepared as described in Example 1.

TABLE 11

| Formulation # | Ingredients |
|---|---|
| Formulation #1 | 0.4% Octreotide Acetate, |
| | 0.15% Methocel 4000 |
| Formulation #2 | 0.4% Octreotide Acetate, |
| | 2% Avicel RC-591 |
| Formulation #3 | 0.4% Octreotide Acetate, |
| | 10% PEG 400 |
| Formulation #4 | 1 mg/mL Sandostatin IR |

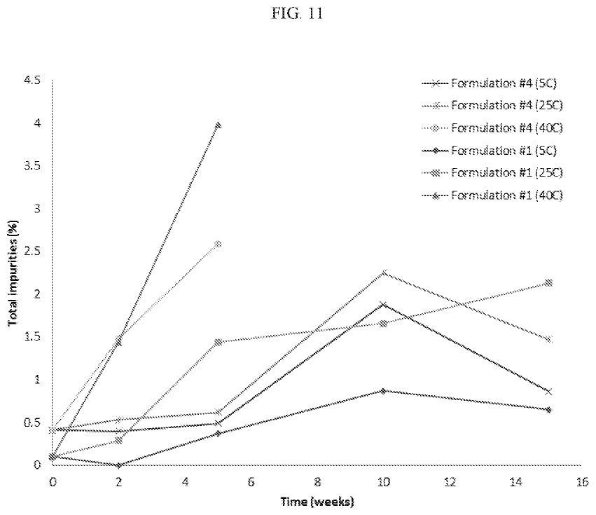

Samples of octreotide formulations in glass vials were stored at 5° C., 25° C./60% relative humidity, and 40° C./75% relative humidity over a period of time. Samples were measured by HPLC for degradation products at certain time points. The percentages of total degradation products identified by HPLC, calculated as % of total AUC for octreotide, are provided in Table 12. The data for Formulations 1 and 4 upon storage at 40, 25, and, 5 degrees Celsius are also shown in FIG. 11.

TABLE 12

| Formulation # | #1 | #2 | #3 | #4 |
|---|---|---|---|---|
| 0 weeks at 5° C. | 0.1 | 0 | 0.44 | 0.41 |
| 2 weeks at 5° C. | 0 | 0.1 | 1.03 | 0.4 |
| 4 weeks at 5° C. | N/A | 0.39 | N/A | N/A |
| 5 weeks at 5° C. | 0.37 | N/A | N/A | 0.49 |
| 10 weeks at 5° C. | 0.87 | N/A | N/A | 1.88 |
| 15 weeks at 5° C. | 0.65 | N/A | N/A | 0.86 |
| 0 weeks at 25° C. | 0.1 | 0 | 0.44 | 0.41 |
| 2 weeks at 25° C. | 0.29 | 0.53 | 5.08 | 0.53 |
| 4 weeks at 25° C. | N/A | 0.87 | N/A | N/A |
| 5 weeks at 25° C. | 1.44 | N/A | N/A | 0.62 |
| 10 weeks at 25° C. | 1.66 | N/A | N/A | 2.25 |
| 15 weeks at 25° C. | 2.13 | N/A | N/A | 1.47 |
| 0 weeks at 40° C. | 0.1 | 0 | 0.44 | 0.41 |
| 2 weeks at 40° C. | 1.44 | 1.26 | 10.27 | 1.48 |
| 4 weeks at 40° C. | N/A | 3.3 | N/A | N/A |
| 5 weeks at 40° C. | 3.98 | N/A | N/A | 2.59 |

### Example 6: Preparation of Lanreotide Compositions

Lanreotide compositions were prepared by the following procedure. A vessel was placed on a mixer, and to the vessel was added 95% of the target amount of water. The target amount of buffering agent was added to the vessel. Then the target amount of excipient was slowly added to the vessel. Mixing is continued until the excipient was visually dissolved. The target amount of lanreotide acetate was added to the vessel, and mixing was continued until the lanreotide acetate was visually dissolved. The target amount of tonicity agent was added to the vessel, and mixing was carried out for 10 minutes. The target amount of DDM that has equilibrated at room temperature for at least 2 hours was added to the vessel, and mixing was continued for at least 25 minutes. The pH of the solution in the vessel was measured and adjusted if necessary. Additional water was added until the target amount of water was reached.

### Example 7: Lanreotide Bioavailability Following Intranasal and Subcutaneous Administration

To determine the plasma pharmacokinetics of lanreotide following administration of lanreotide formulations, three lanreotide formulations as provided in Table 13 were prepared as described in Example 6.

TABLE 13

| Formulation | Ingredients | Physical characterization |
|---|---|---|
| Formulation #1 | Lanreotide (0.25 mg/mL) in | Clear, colorless liquid |
| | 10 mM acetate, 250 mM | essentially free from |
| | mannitol, pH = 5.5 | visible particles |
| Formulation #2 | Lanreotide (4 mg/mL) in | Clear, colorless liquid |
| | 10 mM acetate, 250 mM | essentially free from |
| | mannitol, 0.18% DDM, | visible particles |
| | pH = 5.5 | |
| Formulation #3 | Lanreotide (4 mg/mL) in | Clear, colorless liquid |
| | 10 mM acetate, Methocel | essentially free from |
| | 4000 0.15% w/w, 250 mM | visible particles |
| | mannitol, 0.18% DDM, | |
| | pH = 5.5 | |

Three groups of male and female New Zealand White rabbits were administered the test formulations as summarized in Table 14.

TABLE 14

| | | | | Dose | | Number of |
|---|---|---|---|---|---|---|
| | Route of | | Dose Vol | Concentration | | Animals and |
| Group | Administration | Formulation | (μL) | (mg/mL) | | Sex |
| 1 | Subcutaneous | 1 | 500 μL | 0.25 | mg/mL | 2M/2F |
| 2 | Intranasal | 2 | 100 μL | 4 | mg/mL | 2M/2F |
| | | | (50 μL per | | | |
| | | | nostril) | | | |
| 3 | Intranasal | 3 | 100 μL | 4 | mg/mL | 2M/2F |
| | | | (50 μL per | | | |
| | | | nostril) | | | |

During acclimation, animals were conditioned to intranasal dosing by single installations of 50 μL saline into both nares three times a day for three consecutive days. Groups 2 and 3 were dosed intranasally via instillation of 50 μL into both nares using a Pipetteman and disposable plastic tip. Rabbits in Group 1 were dosed subcutaneously via bolus injection into the nape of the neck using a sterile stainless steel needle attached to a sterile glass syringe. Following dose administration, ten serial blood samples (˜2.0 ml each) were collected by direct venipuncture from a marginal ear vein and deposited into prelabeled 2-ml draw tubes containing K₂EDTA as the anticoagulant at the following times relative to dosing: ˜−30 (predose), 5, 10, 20, 30, 40, 60, 90, 120, and 150 minutes. The total volume collected did not exceed 1% of body weight for the animals.

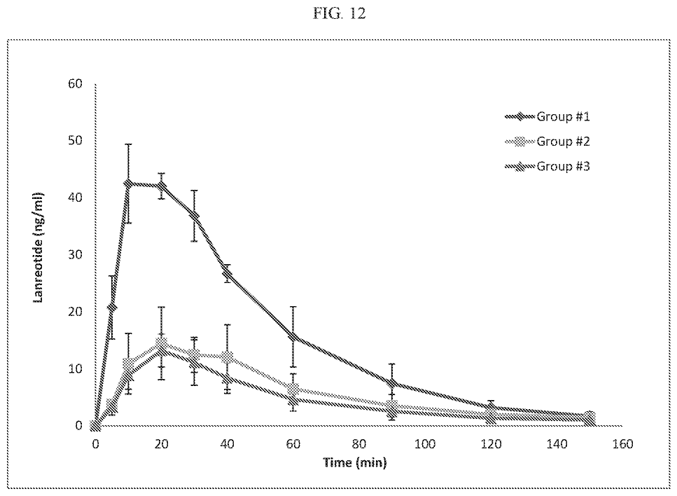

Data analysis consisted of recording and documenting animal body weights, dosing details, animal observations, and blood sampling times (actual and elapsed). Bioanalysis of rabbit plasma samples for lanreotide and pharmacokinetic analysis of the lanreotide plasma concentration-time data are provided in Table 15 and FIG. 12.

TABLE 15

| Group # | #1 | #2 | #3 |
|---|---|---|---|
| 0 | 0 | 0 | 0 |

| | | | | |
|---|---|---|---|---|
| 5 min | (ng/mL) | 20.75 | 3.80 | 3.3 |
| 10 min | (ng/mL) | 42.45 | 10.90 | 8.9 |
| 20 min | (ng/mL) | 42.03 | 14.45 | 13.2 |
| 30 min | (ng/mL) | 36.83 | 12.45 | 11.1 |
| 40 min | (ng/mL) | 26.68 | 12.07 | 8.4 |
| 60 min | (ng/mL) | 15.60 | 6.48 | 4.6 |
| 90 min | (ng/mL) | 7.42 | 3.53 | 2.5 |
| 120 min | (ng/mL) | 3.19 | 2.02 | 1.3 |
| 150 min | (ng/mL) | 1.69 | 1.46 | 1.0 |
| Cₘₐₓ | (ng/mL) | 44.20 | 14.58 | 13.2 |
| AUC | (ng · min/mL) | 2344.40 | 901.14 | 698.94 |

### Example 8: Stability of Lanreotide Formulations

To determine the stability of lanreotide formulations, various lanreotide formulations are prepared as described in Example 6.

Samples of lanreotide formulations in glass vials are stored at 5° C., 25° C./60% relative humidity, and 40° C./75% relative humidity over a period of time. Samples are measured by HPLC for degradation products at certain time points.

### Example 9: Study to Assess the Safety, Tolerability, Pharmacokinetics, and Pharmacodynamics of Octreotide Formulation in Human Subjects

A study to assess the safety, tolerability, pharmacokinetics, and pharmacodynamics of octreotide formulation was conducted in human subjects. A trial formulation (DP1038) containing octreotide acetate, mannitol, Methocel 4000, and DDM was prepared as described in Example 1.

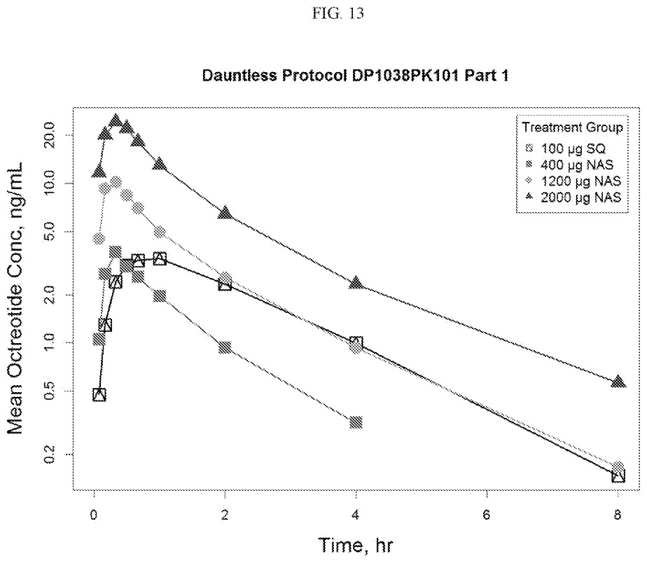

Part 1 of the study evaluated the safety, tolerability, and pharmacokinetics of the trial formulation in a four-way crossover modified Latin square design. As shown in Table 16, each subject in Part 1 of the study received three, single intranasal administrations of the trial formulation (each at a different dose level), plus a single 100 μg subcutaneous administration of Sandostatin Injection, with a one-day washout period between each dose. The three intranasal trial formulation dose levels evaluated in Part 1 were 400 μg, 1200 μg, and 2000 μg. Intranasal trial formulation administrations were performed in a dose-blinded manner. Samples were collected on Days 1, 3, 5, and 7 within 1 hour prior to study drug administration and post-dose at 5 minutes (±2 minutes), 10 minutes (±2 minutes), 20 minutes (±8 minutes), 30 minutes (±2 minutes), 40 minutes (±5 minutes), 1 hour (±5 minutes), 2 hours (±10 minutes), 4 hours (±15 minutes), and 8 hours (±15 minutes). FIG. 13 shows mean plasma concentrations of octreotide following administration of the trial formulation at different dose levels to human subjects.

TABLE 16

| | Day 1 | Day 3 | Day 5 | Day 7 |
|---|---|---|---|---|
| N = 3 | NAS DP1038 | NAS DP1038 | NAS DP1038 | Sandostatin |
| | 400 μg | 1200 μg | 2000 μg | Injection 100 μg |
| N = 3 | NAS DP1038 | NAS DP1038 | Sandostatin | NAS DP1038 |
| | 1200 μg | 400 μg | Injection 100 μg | 2000 μg |
| N = 3 | NAS DP1038 | Sandostatin | NAS DP1038 | NAS DP1038 |
| | 2000 μg | Injection 100 μg | 400 μg | 1200 μg |
| N = 3 | Sandostatin | NAS DP1038 | NAS DP1038 | NAS DP1038 |
| | Injection 100 μg | 2000 μg | 1200 μg | 400 μg |

“NAS” denotes intranasal administration

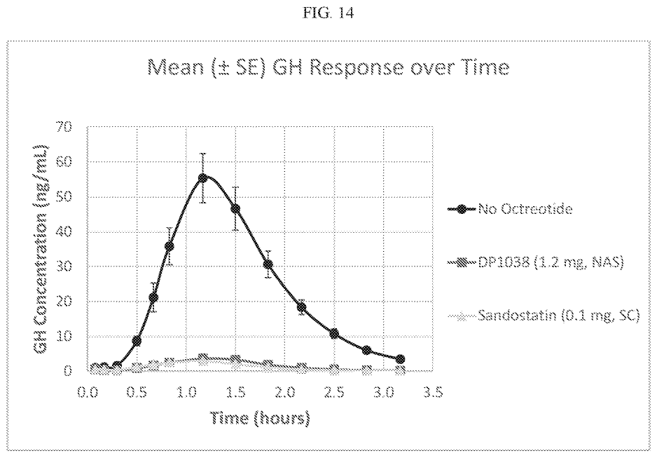

In Part 2 of the study, 20 subjects underwent a standard GHRH/arginine challenge to establish baseline values of GH and IGF-1 levels without any study treatment on Day 1. Half of the subjects were randomized to receive one intranasal dose of the trial formulation on Day 3, to undergo a one-day washout on Day 4, and to receive one subcutaneous dose of Sandostatin Injection on Day 5. The other half of the subjects were randomized to receive drugs in the reverse sequence on the same study days. After each treatment, subjects underwent the GHRH/arginine challenge. Samples were collected on Days 3 and 5 within 1 hour prior to study drug administrations, post-octreotide administration (intranasal or subcutaneous) at 5 minutes (±2 minutes), 10 minutes (±2 minutes), Tₘₐₓ (as determined from Study Part 1; ±10 minutes), 30 minutes (±5 minutes) and 40 minutes (±5 minutes). Additional samples were collected relative to the end of the L-arginine infusion at completion (±5 minutes), and at 20 minutes (±5 minutes), 40 minutes (±5 minutes), 60 minutes (±10 minutes), 80 minutes (±10 minutes), 100 minutes (±10 minutes), 120 minutes (±10 minutes), 140 minutes (±10 minutes), 160 minutes (±10 minutes), 4 hours (±15 minutes), and 8 hours (±15 minutes). FIG. 14 shows growth hormone response in human subjects following administration of the trial formulation and Sandostatin IR.

All documents, including patents, patent application and publications cited herein, including all documents cited therein, tables, and drawings, are hereby expressly incorporated by reference in their entirety for all purposes.

While the foregoing written description of the compositions, uses, and methods described herein enables one of ordinary skill in the art to make and use the compositions, uses, and methods described herein, those of ordinary skill in the art will understand and appreciate the existence of variations, combinations, and equivalents of the specific embodiment, method, and examples herein. The compositions, uses, and methods provided herein should therefore not be limited by the above-described embodiments, methods, or examples, but rather encompasses all embodiments and methods within the scope and spirit of the compositions, uses, and methods provided herein.

1. A pharmaceutical composition comprising:
a) a therapeutic peptide or a pharmaceutically acceptable salt thereof;
b) an alkylsaccharide; and
c) a pharmaceutically acceptable excipient selected from the group consisting of polyethylene glycol of average molecular weight less than about 360 Dalton, alkylcellulose, hydroxyalkyl cellulose, and hydroxyalkyl alkylcellulose.

2. The pharmaceutical composition of claim 1, wherein the therapeutic peptide is cyclic.

3. The pharmaceutical composition of claim 1, wherein the therapeutic peptide is a somatostatin analog or a pharmaceutically acceptable salt thereof.

4. The pharmaceutical composition of claim 1, wherein the therapeutic peptide is selected from the group consisting of lanreotide, octreotide, pasireotide, and pharmaceutically acceptable salts of any of the foregoing.

5. The pharmaceutical composition of claim 1, wherein the therapeutic peptide is octreotide or a pharmaceutically acceptable salt thereof.

6. The pharmaceutical composition of claim 1, wherein the alkylsaccharide is an alkylglycoside.

7. The pharmaceutical composition of claim 1, wherein the alkylsaccharide comprises a C₈-C₁₆ alkyl moiety.

8. The pharmaceutical composition of claim 1, wherein the alkylsaccharide comprises a saccharide selected from the group consisting of maltose, sucrose, and glucose.

9. The pharmaceutical composition of claim 1, wherein the alkylsaccharide consists of a C₁₂ alkyl moiety linked by glycosidic linkage to maltose.

10. The pharmaceutical composition of claim 1, wherein the alkylsaccharide is n-Dodecyl-4-O-α-D-glucopyranosyl-β-D-glucopyranoside (DDM).

11. The pharmaceutical composition of claim 1, wherein the pharmaceutically acceptable excipient is selected from the group consisting of alkylcellulose, hydroxyalkyl cellulose, and hydroxyalkyl alkylcellulose.

12. The pharmaceutical composition of claim 1, wherein the pharmaceutically acceptable excipient is selected from the group consisting of hydroxyethyl cellulose, hydroxylpropyl cellulose, hydroxyethyl methyl cellulose, hydroxypropyl methyl cellulose, ethyl hydroxyethyl cellulose, and methylcellulose.

13. The pharmaceutical composition of claim 1, wherein the pharmaceutically acceptable excipient is Methocel 4000.

14. The pharmaceutical composition of claim 1, wherein the pharmaceutically acceptable excipient is polyethylene glycol of average molecular weight less than about 360 Dalton.

15. The pharmaceutical composition of claim 14, wherein the average molecular weight of the polyethylene glycol is from 100 to 300 Daltons.

16. The pharmaceutical composition of claim 14, wherein the polyethylene glycol is PEG 200.

17. The pharmaceutical composition of claim 1, further comprising a tonicity agent.

18. The pharmaceutical composition of claim 17, wherein the tonicity agent is mannitol.

19. The pharmaceutical composition of claim 1, further comprising a buffering agent.

20. The pharmaceutical composition of claim 19, wherein the pH of the composition is from 3 to 8.

21. The pharmaceutical composition of claim 19, wherein the pH is of the composition is from 4.5 to 6.

22. The pharmaceutical composition of claim 1, wherein the concentration of the therapeutic peptide is from 0.01% (w/w) to 10% (w/w).

23. The pharmaceutical composition of claim 1, wherein the concentration of the therapeutic peptide is from 0.1% (w/w) to 3% (w/w).

24. The pharmaceutical composition of claim 1, wherein the concentration of the alkylsaccharide is from 0.01% (w/w) to 5% (w/w).

25. The pharmaceutical composition of claim 1, wherein the concentration of the alkylsaccharide is from 0.1% (w/w) to 1% (w/w).

26. The pharmaceutical composition of claim 1, wherein the concentration of the pharmaceutically acceptable excipient is from 0.01% (w/w) to 5% (w/w).

27. The pharmaceutical composition of claim 1, wherein the concentration of the pharmaceutically acceptable excipient is from 0.01% (w/w) to 1% (w/w).

28. The pharmaceutical composition of claim 17, wherein the concentration of the tonicity agent is from 0.01% (w/w) to 10% (w/w).

29. The pharmaceutical composition of claim 17, wherein the concentration of the tonicity agent is from 0.01% (w/w) to 6% (w/w).

30. A method of treating a condition selected from the group consisting of acromegaly, carcinoid tumors, vasoactive intestinal peptide secreting tumors, diarrhea associated with acquired immune deficiency syndrome (AIDS), diarrhea associated with chemotherapy, diarrhea associated with radiation therapy, dumping syndrome, adrenal gland neuroendocrine tumors, bowel obstruction, enterocutaneous fistulae, gastrinoma, acute bleeding of gastroesophageal varices, islet cell tumors, lung neuroendocrine tumors, malignancy, meningiomas, gastrointestinal tract neuroendocrine tumors, thymus neuroendocrine tumors, pancreatic fistulas, pancreas neuroendocrine tumors, pituitary adenomas, short-bowel syndrome, small or large cell neuroendocrine tumors, thymomas and thymic carcinomas, Zollinger Ellison syndrome, acute pancreatitis, breast cancer, chylothorax, congenital lymphedema, diabetes mellitus, gastric paresis, hepatocellular carcinoma, non-variceal upper gastrointestinal bleeding, obesity, pancreaticoduodenectomy, prostate cancer, protein-losing enteropathy, small cell lung cancer, thyroid cancer, thyroid eye disease, vascular (arterio-venous) malformations of the gastrointestinal tract, polycystic kidney disease, Cushing's disease, GHRH-producing tumors, and other conditions resulting in abnormally elevated growth hormone, insulin, or glucagon levels in an individual in need thereof, the method comprising administering to the individual a pharmaceutical composition of claim 1.

31. The method of claim 30, wherein the pharmaceutical composition is administered orally, sublingually, subcutaneously, intravenously, intranasally, topically, transdermally, intraperitoneally, intramuscularly, intrapulmonarilly, vaginally, rectally, or intraocularly.

32. The method of claim 30, wherein the pharmaceutical composition is administered intranasally.
